(19) United States
(12) Patent Application Publication
(10) Pub. No.: US 2003/0089885 A1
(43) Pub. Date: May 15, 2003

(54) Use of low molecular weight acetal, alcohol, acylated alcohol and ester compounds to block or reduce odor of carboxylic acids

(75) Inventors: Dan Rogers, San Diego, CA (US); Craig Warren, San Diego, CA (US); Ming Qi, San Diego, CA (US)

Correspondence Address: PILLSBURY WINTHROP, LLP, P.O. BOX 10500, MCLEAN, VA, 22102, US

(73) Assignee: Senomyx, Inc., La Jolla, CA

(21) Appl. No.: 10/127,563

(22) Filed: Apr. 23, 2002

Related U.S. Application Data
(60) Provisional application No. 60/286,003, filed on Apr. 25, 2001.
(60) Provisional application No. 60/294,261, filed on May 31, 2001.
(60) Provisional application No. 60/348,644, filed on Jan. 17, 2002.
(60) Provisional application No. 60/348,645, filed on Jan. 17, 2002.
(60) Provisional application No. 60/348,675, filed on Jan. 17, 2002.

(51) Int. Cl. C09K 3/00; A61L 9/01

(52) U.S. Cl. 252/183.11; 424/76.1

(57) ABSTRACT

Low molecular weight acetal, alcohol, O-acylated alcohol and ester compounds are identified that block the odor of specific carboxylic acids especially propionic acid, butyric acid, isovaleric acid 3-methyl-2-hexenoic acid and hexanoic acid. These compounds exhibit surprisingly high odor blocking properties and are therefore useful additives to compositions such as: a) personal care products including underarm deodorant and antiperspirant, hair shampoo and conditioner, toilet soap, and skin lotion; b) fabric care products including laundry detergents, fabric conditioners, and fabric fresheners; c) pet care products including deodorant, shampoo, conditioner, and d) home care products including air fresheners, carpet fresheners, surface cleaners and hand wash dish detergent.

### RELATED APPLICATIONS

This claims priority to U.S. Provisional U.S. Serial No. 60/286,003 filed Apr. 25, 2001, U.S. Provisional U.S. Serial No. 60/294,261, filed May 31, 2001, U.S. Serial No. 60/348,644 filed Jan. 17, 2002, U.S. Serial No. 60/348,645 filed Jan. 17, 2002, and U.S. Serial No. 60/348,675 filed Jan. 17, 2002 all of which are incorporated by reference in there entirety herein.

### BACKGROUND OF THE INVENTION

1. Field of the Invention

It is generally known that the malodor of human and animal bodily secretions, particularly sweat, is attributable in part to the presence of unpleasant smelling carboxylic acids. These acids include propionic acid (a component of foot malodor), hexanoic acid (a component of pet malodor), and 3-methyl-2-hexenoic acid and isovaleric acid (both components of human underarm malodor). (Labows, Reilly et al. 1999)

In order to counteract such odors, numerous commercial products are available, for example: a) personal care products including but not limited to underarm deodorants and antiperspirants, perfume, toilet soap, hair shampoo and conditioner, and skin lotion; b) fabric care products including but not limited to laundry detergents, fabric conditioners, and fabric fresheners; c) pet care products including but not limited to pet deodorant, shampoo and conditioner and, d) home care products including but not limited to air fresheners, carpet fresheners, hand dish wash detergents, and surface cleaners. These products contain a variety of ingredients that are intended to mask malodor. A key malodor masking ingredient is fragrance which masks the malodor either by overpowering it or blending with it to make it more pleasant.

Conventional deodorant compositions include a variety of fragrance materials that provide a pleasant perfume that masks the malodor compound generally by one of two mechanisms. In the first mechanism, the masking fragrance blends with the malodor compound to provide a different and more desirable aroma. In the second mechanism, the masking fragrance is employed in a large quantity to overwhelm the malodor compound.

Unfortunately both types of masking mechanisms have serious disadvantages. Neither mechanism completely eliminates the perception of malodor. Thus there is a tendency to use increasing amounts of deodorant composition to completely eliminate the perception of malodor. Furthermore, the masking effect is an additive effect and so the total odor level is increased. Even though the fragrances used may be very pleasant at low concentration, the total odor level at the relatively high concentrations required to achieve moderate masking of the malodor may itself be offensive.

U.S. Pat. No. 4,622,221 (Schleppnik) discloses a method of counteracting a malodor in air. The method comprises introducing into the air an effective amount of cyclohexyl-1-ethyl-n-butyrate, and derivatives thereof.

U.S. Pat. No. 5,589,158 (Mankoo) discloses a flavor enhancing composition comprising between about 30% and 90% by weight benzyl benzoate and between about 3% and 15% by weight neryl acetate. European patent application no. 0 251 542 (Lion Corporation) discloses an oral composition comprising a zinc salt and 0.1% to 5% by weight of polyoxyethylene hydrogenated castor oil. However, neither of these patents relates to the use of malodor masking compounds that function comparably to those of the present invention. In particular, the compositions of Markoo do not effectively block breath odor. Also, the Lion patent is directed to the use of zinc salts that react with mercaptans that function via an entirely different mechanism than the inventive compounds.

Also, Eur. Pat. Appl. 1113105 (Marano, F. A., J. T. Van Elst, et al. (2001) disclose that compositions created from beta-naphthyl Me ether, Me beta-naphthyl ketone; benzyl acetone; a mixt. Of hexahydro-4, 7-methanoinden-5-yl propionate and hexahydro-4,7-methanoinden-6-yl propionate; gamma-Me ionone; geranonitrile (3,7-dimethyl-2,6-octadiene-1-nitrile); dodecahydro-3a, 6, 6, 9a-tetramethylnaphtho (2,1-b)furan, ethylene glycol cyclic ester of n-dodecanedioic acid, and 1-cyclohexadecen-6-one and/or 1-cycloheptadecen-10-one mask human sweat malodor.

Canadian patent no. 987,597 (McNamara et al.) discloses compositions having odor masking properties. The compositions comprise a compound selected from the group consisting of α-ionone, α-methyl ionone, citral, geranyl formate, and geranyl acetate. These compounds are used in an amount from 50 to 3000 parts per million, by weight.

While the above compositions provide some degree of freshening action, none of the above compositions are satisfactory malodor counteractant compositions.

Another way to counteract a matodor is to suppress or to eliminate it by a phenomenon called cross adaptation in which sniffing one odorant alters one's sensitivity to other odorants. Odorants similar in structure are more likely to exhibit cross adaptation than structural dissimilar ones (Cain 1974; Cain 1975; Cain and Polack 1992). Examples of cross adaptation between odorants similar in structure are reported by Preti and Pierce who found that the methyl and ethyl esters of 3-methyl-2-hexenoic acid cross-adapt with its sweaty smelling parent, 3-methyl-2-hexenoic acid to lower its perceived odor intensity by around 25%. (Pierce, Blank et al. 1996; Preti, Pierce et al. 1996; Pierce, Blank et al. 1998).

Examples of cross adaptation between molecules dissimilar in structure are rare and the level of reduced olfactory sensitivity to the target molecule due to cross adaptation rarely exceeds 30% (Cain and Polack 1992). By contrast, the present invention involves the discovery and use of three families of compounds: a) low molecular weight acetals, b) low molecular weight esters, and c) low molecular weight alcohols that reduce human olfactory sensitivity to carboxylic acids associated with pet and human malodor by as much as 97%.

Therefore, notwithstanding what has been reported there exists a need for improvements with respect to identifying compounds that actually have odor reducing properties. In particular, there exists a need for compounds that block the perception of a malodor by cross adaptation in which a single molecule or a mixture of molecules reduce or eliminate the perception of the malodor. The alternative, which is the current practice, is the use of odorants that either overpower the malodor or change its odor character.

### OBJECTS OF THE INVENTION

Thus, it is an object of this invention to identify specific compounds that effectively block the malodor of specific carboxylic acids by cross-adaptation.

More specifically, it is an object of this invention to identify particular low molecular weight acetals, esters of carboxylic acids and alcohols and their corresponding O-acylated derivatives that block or reduce human olfactory sensitivity to volatile, low molecular weight carboxylic acids such as butyric acid, propionic acid, hexanoic acid, isovaleric acid, and 3-methyl-2-hexenoic acid.

Even more specifically, it is an object of the invention to identify: a) acetals of isovaleraldehyde and 2-methyl-cyclopropyl-3-carboxaldehyde, b) low molecular weight alcohols, especially C3-C12 saturated and unsaturated aliphatic alcohols, as well as alcohols containing C3-C8 cycloalkyl side chains and their O-acylated derivatives; and c) alkyl esters of isovaleric acid and cyclopropyl and cyclobutyl carboxylic acid alkyl esters that substantially reduce the odor of propionic, butyric, isovaleric, 3-methyl-2-hexenoic, and hexanoic acids by cross-adaptation.

It is another object of the invention to provide compositions comprising an amount of at least one low molecular weight acetal, alcohol, O-acylated alcohol or ester compound that blocks or reduces human sensitivity to the malodor of butyric, isovaleric, 3-methyl-2-hexenoic acid or hexanoic acid.

It is an even more specific object of the invention to produce: a) personal care products including but not limited to underarm deodorants and antiperspirants, toilet soaps, hair shampoo and conditioner, and skin lotions; b) fabric care products including but not limited to laundry detergents, fabric conditioners, and fabric fresheners; c) pet care products including but not limited to pet deodorants, shampoo and conditioner, and d) home care products including but not limited air fresheners, carpet fresheners and surface cleaners that contain as an active malodor counteractant ingredient at least one of the following: a) acetals of isovaleraldehyde and 2-methyl-cyclopropyl-3-carboxaldehyde, b) low molecular weight alcohols, especially C3-C12 saturated and unsaturated aliphatic alcohols, as well as alcohols containing C3-C8 cycloalkyl side chains and their O-acylated derivatives; and c) alkyl esters of isovaleric acid and cyclopropyl and cyclobutyl carboxylic acid alkyl esters that substantially reduce the odor of propionic, butyric, isovaleric, 3-methyl-2-hexenoic, and hexanoic acids by cross-adaptation.

In preferred embodiments, the low molecular weight acetal, alcohol, O-acylated alcohol or ester compounds will block the perception of a carboxylic acid selected from isovaleric acid, butyric acid, 3-methyl-2-hexenoic acid or hexanoic acid by at least 60%, more preferably at least 70%, and even more preferably by at least 90%.

### BRIEF DESCRIPTON OF THE FIGURE

FIG. 1 contains the Labeled Magnitude scale used to quantitatively measure the odor intensity changes reported herein.

### DETAILED DESCRIPTION OF THE INVENTION

The present invention resides in the discovery that certain low molecular weight acetal, alcohol, O-acylated alcohol and ester compounds significantly cross-adapt with certain low molecular weight carboxylic acids, especially isovaleric acid, a malodor constituent of human axillary secretions. Particularly, as measured by the Malodor Reduction Test which is presented in Example 9, acetal, alcohol, O-acylated alcohol or ester compound compounds have been identified that block perceived odor intensity by 60% or more. These results are truly surprising as they exceed the levels of blocking reported in the literature for compounds reported to block the odor of isovaleric acid. Thus, these compounds should be well suited for use in malodor counteractant compositions and for usage in ambient environments subject to the accumulation of low molecular weight malodorous carboxylic acids.

Sniffing an effective amount of the low molecular weight acetal, alcohol, O-acylated alcohol or ester compound of the present invention significantly reduces human olfactory sensitivity to carboxylic acids. This is a specific example of the phenomenon known as cross-adaptation where it is observed that sniffing an odorant alters sensitivity to other odorants. The low molecular weight acetal, alcohol, O-acylated alcohol or ester compound reported herein were found to block the perception of isovaleric acid when both the compound and isovaleric acid are sniffed in the same nostril. To the best of the inventors' knowledge, these findings are novel.

Using the malodor reduction test presented in Example 9, a number of acetal, alcohol, O-acylated alcohol or ester compounds were found to significantly reduce the perceived intensity of isovaleric acid. The observed malodor reduction percentages observed for such compounds are presented in Tables 1-3 in Example 9. It can be seen there from that all of the represented compounds reduce the malodor of isovaleric acid by at least 67% and as high as 97%. As stated previously, these results are truly unexpected based on known compounds that block the odor of isovaleric acid.

It is anticipated based on the results observed with the exemplified acetal, alcohol, O-acylated alcohol or ester compounds that similar results will be achieved with related compounds. Moreover, it is anticipated that such compounds will inhibit the odor of other malodorous carboxylic acids, particularly butyric acid, propionic acid, hexanoic acid, and 3-methyl-2-hexenoic acid.

It is anticipated further that higher molecular weight acetal, alcohol, O-acylated alcohol or ester compounds having calculated octanol-water partition coefficients (cLogP) of three or higher, in addition to blocking or reducing the perception of the unpleasant smelling carboxylic acid, will also be substantive to skin and cloth and consequently should be useful as additives in bar soaps, laundry detergents, foot and underarm deodorants, and other deodorizing compositions suitable for application to the skin or cloth, and other substrates prone to contamination by such carboxylic acids.

It is especially anticipated based on the results described herein that: a) acetals of isovaleraldehyde and 2-methyl-cyclopropyl-3-carboxaldehyde, b) low molecular weight alcohols, especially C3-C12 saturated and unsaturated aliphatic alcohols, as well as alcohols containing C3-C8 cycloalkyl side chains and their O-acylated derivatives; and c) alkyl esters of isovaleric acid and cyclopropyl and cyclobutyl carboxylic acid alkyl esters will, in addition to isovaleric acid (human axillary secretion constituent) and butyric acid (baby vomit constituent), block the odor of propionic acid (a constituent of foot odor), hexanoic acid (pet odor constituent) and 3-methyl-2-hexenoic acid (human axillary secretion constituent).

Thus, the subject invention provides particular classes of acetal, alcohol, O-acylated alcohol compounds that block the odor of unpleasant smelling carboxyic in acids, especially butyric acid, propionic acid, isovaleric acid, hexanoic acid and 3-methyl-2 hexenoic acid.

In one embodiment, acetal compounds according to the invention will possess in the general structure (I) below:

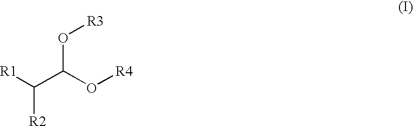

wherein R1 is selected from the group consisting of

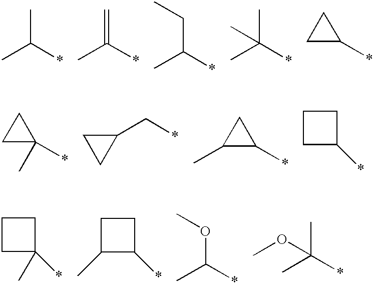

R2 is selected from the group consisting of H, Me, Et, OH, and OMe:

R3 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂OCH₃, —CH₂CH₂OCH₂CH₃;

R4 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂OCH₃, —CH₂CH₂OCH₂CH₃;

Preferred examples of such compounds include the following:

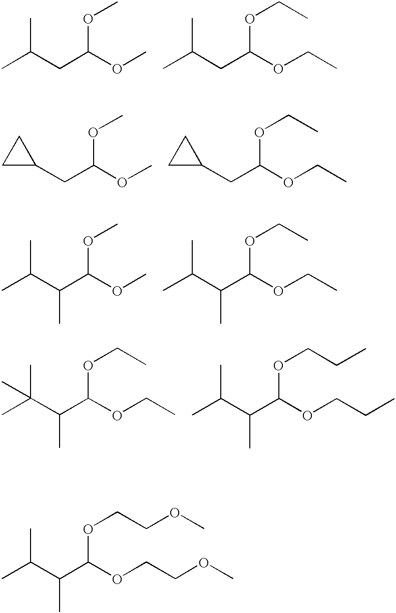

In a second embodiment, acetal components according to the invention can be defined by structures II, III, IV, and V below:

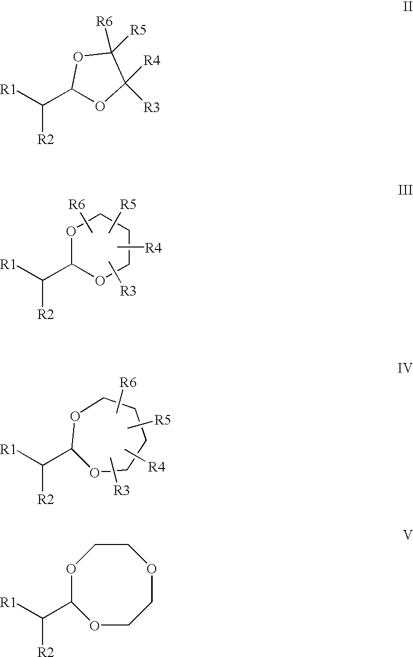

wherein, R1 is selected from

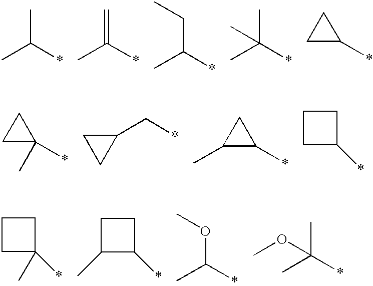

R2 is selected from the group consisting of H, Me, Et, OH and OMe;

R3 represents unsubstitution (i.e., H), or is selected from small normal and branched alkanes and alkenes (C1-10 alkyls), including methyl, ethyl, propyl, isopropyl, —CH₂CH═CH₂, butyl, isobutyl, pentyl, etc.; cycloalkyl (e.g., cyclopropyl, cyclobutyl, cyclopentyl and cyclohexyl); and heteratom substituted alkanes and alkenes, including —CH₂OH, —CH₂OCH₃, —CH₂OCH₂CH₃, —CH₂SCH₃, and —C(O)OCH₃.

R4 represents unsubstitution (i.e., H), or is selected from small normal and branched alkanes and alkenes (C₁₋₅ alkyls and alkenes), including methyl, ethyl, propyl, isopropyl, —CH₂CH═CH₂, butyl, isobutyl, pentyl, etc.; cycloalkyl (e.g., cyclopropyl, cyclobutyl, cyclopentyl and cyclohexyl); and heteratom substituted alkanes and alkenes, including —CH₂OH, —CH₂OCH₃, —CH₂OCH₂CH₃, —CH₂SCH₃, and —C(O)OCH₃.

R5 represents unsubstitution (i.e., H), or is selected from small normal and branched alkanes and alkenes (C₁₋₅ alkyls and alkenes), including methyl, ethyl, propyl, isopropyl, —CH₂CH═CH₂, butyl, isobutyl, pentyl, etc.; cycloalkyl (e.g., cyclopropyl, cyclobutyl, cyclopentyl and cyclohexyl); and heteratom substituted alkanes and alkenes, including —CH₂OH, —CH₂OCH₃, —CH₂OCH₂CH₃, —CH₂SCH₃, and —C(O)OCH₃.

R6 represents unsubstitution (i.e., H), or is selected from small normal and branched alkanes and alkenes (C₁₋₅ alkyls and alkenes), including methyl, ethyl, propyl, isopropyl, —CH₂CH═CH₂, butyl, isobutyl, pentyl, etc.; cycloalkyl (e.g., cyclopropyl, cyclobutyl, cyclopentyl and cyclohexyl); and heteratom substituted alkanes and alkenes, including —CH₂OH, —CH₂OCH₃, —CH₂OCH₂CH₃, —CH₂SCH₃, and —C(O)OCH₃.

Alternatively, R3 and R4 can be combined to form a carbonyl (═O), or olefin (C═CH₂), or spirocyclic derivative, including spirocyclopropyl, spirocyclobutyl, spirocyclopentyl and spirocyclohexyl.

Still alternatively, R4 and R5 can be combined to form a fused cyclic derivative, including —CH₂-(cyclopropyl), —CH₂CH₂-(cyclobutyl), —CH₂CH₂CH₂-(cyclopentyl), —CH₂CH₂CH₂CH₂-(cyclohexyl), —C(O)OCH₂—, etc;

Specific examples of such acetals are set forth below:

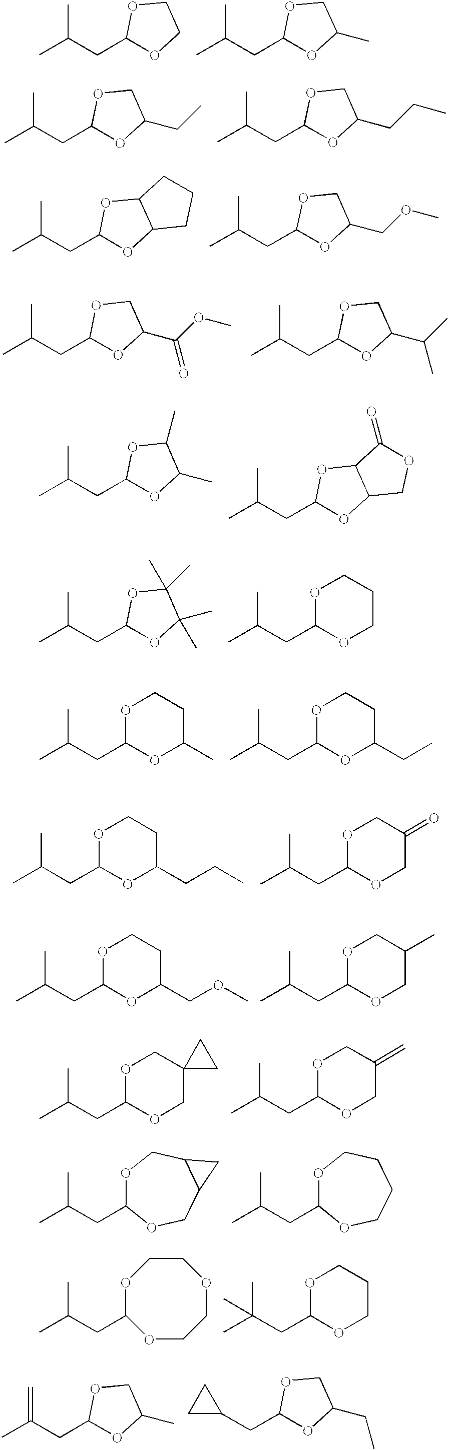

In a third embodiment, acetal compounds according to the invention will comprise substituted cyclopropyl or cyclobutyl-substituted 1 ,3-dioxacycles having one of the following general structures:

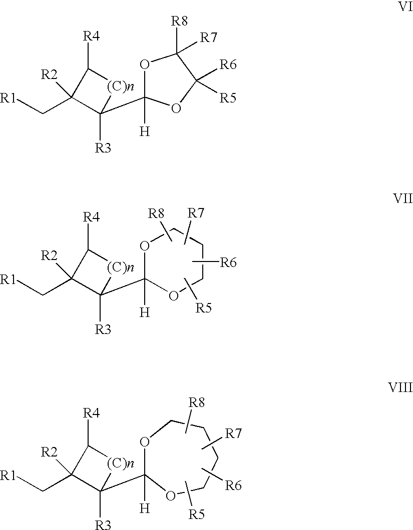

wherein R1 is selected from a group consisting of H, Me and methylene (═CH₂);

R2 is H, or methyl;

R3 is H, methyl, or methoxy (OMe);

R4 is H or methyl;

n is 0 or 1;

R5 represents unsubstitution (i.e., H), or is selected from small normal and branched alkanes and alkenes (C₁₋₅ alkyls and alkenes), including methyl, ethyl, propyl, isopropyl, —CH₂CH═CH₂, butyl, isobutyl, pentyl, etc.; cycloalkyl (e.g., cyclopropyl, cyclobutyl, cyclopentyl and cyclohexyl); and heteratom substituted alkanes and alkenes, including —CH₂OH, —CH₂OCH₃, —CH₂OCH₂CH₃, —CH₂SCH₃, and —C(O)OCH₃.

R6 represents unsubstitution (i.e., H), or is selected from small normal and branched alkanes and alkenes (C₁₋₅ alkyls and alkenes), including methyl, ethyl, propyl, isopropyl, —CH₂CH═CH₂, butyl, isobutyl, pentyl, etc.; cycloalkyl (e.g., cyclopropyl, cyclobutyl, cyclopentyl and cyclohexyl); and heteratom substituted alkanes and alkenes, including —CH₂OH, —CH₂OCH₃, —CH₂OCH₂CH₃, —CH₂SCH₃, and —C(O)OCH₃.

R7 represents unsubstitution (i.e., H), or is selected from small normal and branched alkanes and alkenes (C₁₋₅ alkyls and alkenes), including methyl, ethyl, propyl, isopropyl, —CH₂CH═CH₂, butyl, isobutyl, pentyl, etc.; cycloalkyl (e.g., cyclopropyl, cyclobutyl, cyclopentyl and cyclohexyl); and heteratom substituted alkanes and alkenes, including —CH₂OH, —CH₂OCH₃, —CH₂OCH₂CH₃, —CH₂SCH₃, and —C(O)OCH₃.

R8 represents unsubstitution (i.e., H), or is selected from small normal and branched alkanes and alkenes (C₁₋₅ alkyls and alkenes), including methyl, ethyl, propyl, isopropyl, —CH₂CH═CH₂, butyl, isobutyl, pentyl, etc.; cycloalkyl (e.g., cyclopropyl, cyclobutyl, cyclopentyl and cyclohexyl); and heteratom substituted alkanes and alkenes, including —CH₂OH, —CH₂OCH₃, —CH₂OCH₂CH₃, —CH₂SCH₃, and —C(O)OCH₃.

Alternatively, R5 and R6 can be combined to form carbonyl (═O), or olefin (C═CH₂), or spirocyclic derivatives, including spirocyclopropyl, spirocyclobutyl, spirocyclopentyl and spirocyclohexyl, etc.

Also alternatively, R6 and R7 can be combined to form fused cyclic derivatives, including —CH₂-(cyclopropyl), —CH₂CH₂-(cyclobutyl), —CH₂CH₂CH₂-(cyclopentyl), —CH₂CH₂CH₂CH₂-(cyclohexyl), and —C(O)OCH₂—.

Specific examples thereof include the following compounds:

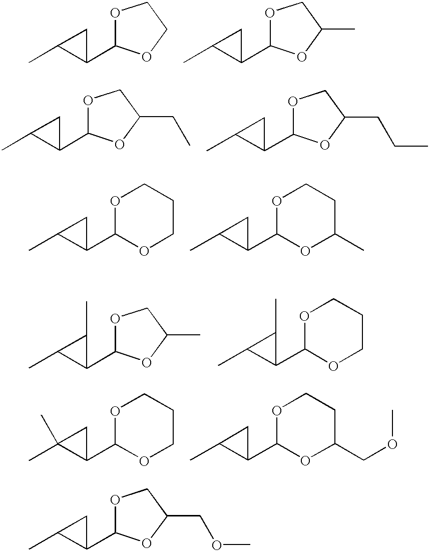

Another example of acetal compounds according to the invention comprises substituted cyclopropyl or cyclobutyl-substituted O-alkyl acetal compounds which are defined by having generic formula IX below:

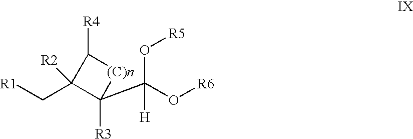

wherein R1 is selected from a group consisting of H, Me and methylene (═CH₂);

R2 is H, or methyl;

R3 is H, methyl, or methoxy (OMe);

R4 is H or methyl;

n is 0 or 1;

R5 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂OCH₃, and —CH₂CH₂OCH₂CH₃; and

R6 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂OCH₃, and —CH₂CH₂OCH₂CH₃.

Examples of such compounds are set forth below:

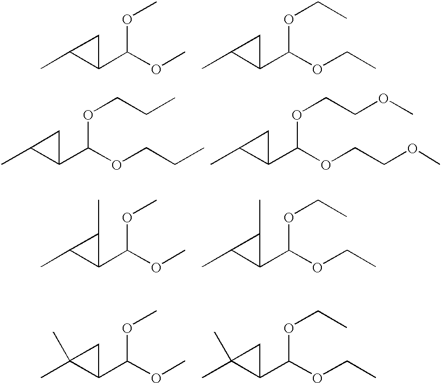

In preferred embodiments, acetal compounds according to the invention will be selected from Table 1: a) 2-Isobutyl-[1,3]dioxane, b) 2-Isobutyl-4-methyl-[1,3]dioxane, c) 2-Isobutyl-4,5-dimethyl-[1,3]dioxolane, d) 2-Isobutyl-4-methyl-[1,3]dioxolane, e) 1 ,1-Dimethoxy-3-methyl-butane, f ) 2-Isobutyl-[1,3]dioxolane, g) 2-(2-Methyl-cyclopropyl)-[1,3]dioxolane, h) 2-Isobutyl-5-methyl-[1,3]dioxane, 1) 1,1-Diethoxy-3-methyl-butane, j) 4-Ethyl-2-isobutyl-[1,3]dioxolane, k) 2-Isobutyl-4-isopropyl-[1,3]dioxolane, l) 2-Isobutyl-4-propyl-[1,3]dioxolane, m) 2-Isobutyl-5-methylene-[1,3]dioxane, n) 2-Isobutyl-tetrahydro-cyclopenta[1,3]dioxole, o) 2-Isobutyl-4,4,5,5-tetramethyl-[1,3]dioxolane, and p) 2-Isobutyl-4-methoxymethyl-[1,3]dioxolane.

In a fourth embodiment, odor-blocking esters of the present invention will produce the general formula (I) set forth below:

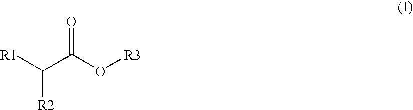

wherein R1 is selected from the group consisting:

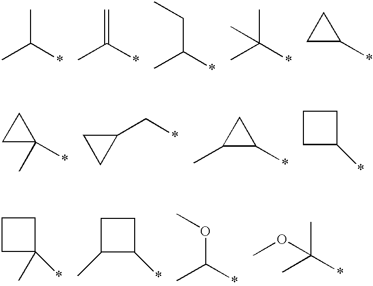

R2 is selected from the group consisting of H, Me, Et, OH, and OMe;

R3 is selected from the group consisting of H, Me, Et, propyl, isopropyl, —CH₂cyclopropyl, —CH₂CH₂CH═CH₂, —CH₂CH₂OCH₃, and —CH₂CH₂OCH₂CH₃.

Examples of such compounds are shown below:

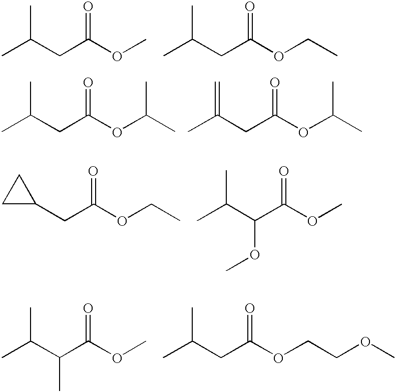

Alternatively, odor blocking esters according to the invention will comprise substituted cyclopropyl or cyclobutyl carboxylic or cyclobutyl carboxylic acid esters of formula II below:

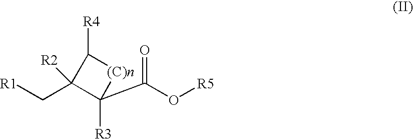

wherein, R1 is H or Me;

R2 is H or Me;

R3 is selected from the group consisting of H, Me, and OMe;

R4 is H or Me;

R5, is selected from the group consisting of H, Me, Et, propyl, isopropyl, —CH₂cyclopropyl, —CH₂CH₂CH═CH₂, —CH₂CH₂OCH₃, —CH₂CH₂OCH₂CH₃, and

n is 0 or 1.

Specific examples thereof include the following compounds:

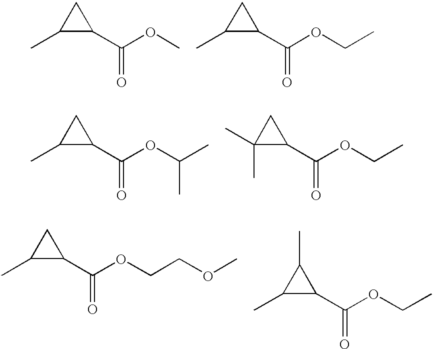

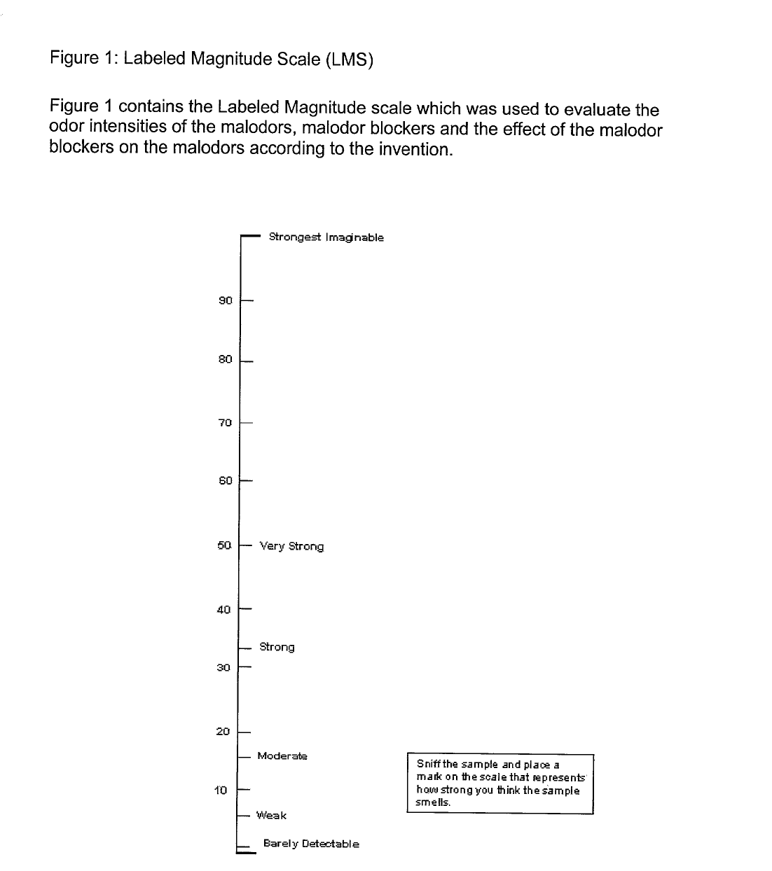

Preferred compounds according to the inventor are those recited in FIG. 1, i.e., ethyl isovalerate, methyl isovalerate, 2-methylcyclopropanecarboxylic acid methyl ester, n-propyl isovalerate, 2-methylcyclopropanecarboxylic acid ethyl ester, isopropyl isovalerate, 2-methylcyclopropanecarboxylic acid isopropyl ester, and 2-methylcyclopropanecarboxylic acid n-propyl ester.

In a fifth embodiment, examples of specific branched alcohols and their O-acyl derivatives according to the invention will have the following general structure (I):

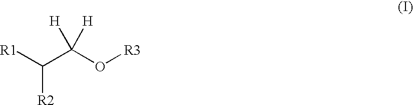

wherein R1 is selected from the group consisting:

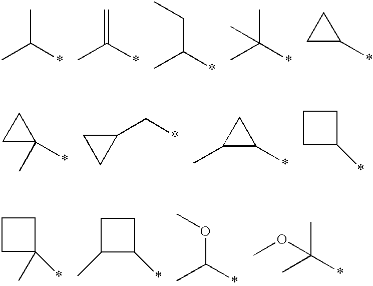

R2 is selected from the group consisting of H, Me, Et, OH, and OMe;

R3 is selected from the group consisting of H, —C(O)R₄, where R4 is selected from Me, Et, Pr, iPr, Bu, and —CH₂cyclopropyl, —CH₂CH₂CH═CH₂, —CH₂OCH₃, —CH₂COCH₂CH₃, —CH₂CH₂OCH₃, and —CH₂CH₂OCH₂CH₃.

Specific examples of such compounds will possess the following structures:

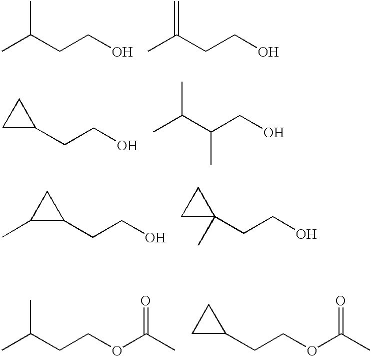

In a sixth embodiment, alcohols and their O-acylated derivatives according to the invention can be checked by the following generic structure (II):

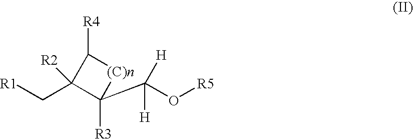

wherein, R1 is H or Me;

R2 is H or Me;

R3 is selected from the group consisting of H, Me, and OMe;

R4 is H or Me;

R5, is selected from the group consisting of H; —C(O)R6, where R6 is selected from the group consisting is selected from Me, Et, Pr, iPr, Bu, and —CH₂cyclopropyl, —CH₂CH₂CH═CH₂, —CH₂OCH₃, —CH₂COCH₂CH₃, —CH₂CH₂OCH₃, —CH₂CH₂OCH₂CH₃, and n is 0 or 1.

Specific examples of such compounds are set forth below:

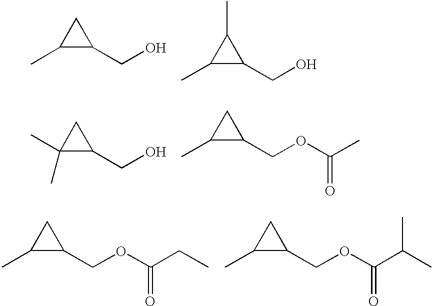

In preferred embodiments, the alcohols and their O-acylated derivatives according to the invention will include (2-methylcyclopropyl)methanol, 2-(2-methylcyclopropyl)ethanol, 2-cyclopropyl ethanol, isoamyl alcohol, 2-(1-methylcyclopropyl)ethanol, isoamyl acetate, (2,2-dimethylcyclopropyl)methanol and 3-methyl but-3-enol.

The compounds according to the present invention will be utilized as active malodor counteractant agents in compositions that are intended to eliminate or reduce the perception of malodorous carboxylic acids such as isovaleric acid, butyric acid, propionic acid, hexanoic acid, and 3-methyl-2-hexenoic acid or in compositions that come in contact with substrates, e.g., human or animal skin, rugs, fabrics, that are prone to the accumulation of such carboxylic acids.

Examples thereof include: a) personal care products including but not limited to deodorants, toilet soap, and skin lotions, b) fabric care products including but not limited to laundry detergents, fabric conditioners, and fabric fresheners; c) pet care products including but not limited to pet deodorants, dog shampoo, dog hair conditioner; and d) home care products including but not limited air fresheners, carpet fresheners and surface cleaners.

These acetal, alcohol, O-acylated alcohol and ester compounds may be used alone or in combination, and may be used in combination with other types of odor reducers.

The amount of such compound or compounds added to such compositions will be an amount that effectively reduces, i.e., by at least 60% or greater, more preferably at least 70% or greater, and still more preferably by at least 80% or greater the odor that would otherwise be perceptible to a subject by a particular acid malodor, i.e., isovaleric or butyric acid. These amounts may be varied dependent on the pertinent environment that the composition is to be used. Typical amounts of compounds according to the invention will range from about 0.01% to 10.0% by weight, more preferably from about 0.1% to 5% by weight and most preferably from about 0.5% to 3% by weight.

Deodorant compositions containing compounds according to the invention can be formulated with other deodorant active ingredients, e.g., antimicrobials, (bactericides, fungicides) malodor-absorbing molecules, and/or perfume chemicals that mask odors or that otherwise provide a desired fragrance. The concentration of active compounds according to the invention will range from 0.01% to 10% by weight, more preferably from about 0.1% to 5% by weight of the composition.

Specific examples of other active ingredients include: a) fragrance ingredients that mask underarm malodor, e.g., (beta-naphthyl Me ether, Me beta-naphthyl ketone; benzyl acetone; a mixt. of hexahydro-4,7-methanoinden-5-yl propionate and hexahydro-4,7-methanoinden-6-yl propionate; gamma-Me ionone; geranonitrile (3,7-dimethyl-2,6-octadiene-1-nitrile); dodecahydro-3a,6,6,9a-tetramethylnaphtho(2,1-b)furan, ethylene glycol cyclic ester of n-dodecanedioic acid, and 1-cyclohexadecen-6-one and/or 1-cycloheptadecen-10-one) (Marano, Van Elst et al. 2001); b) antimicrobials used in underarm deodorants and soaps [2,4,4′-trichloro-2′hydroxy diphenyl ether (triclosan), 3,4,4′-trichlorocarbanilide (triclocarban)]; c) antiperspirant ingredients (aluminum chlorhydrate, aluminum-zirconyl hydroxychloride complex); and d) germicides used in surface cleaners (cetyl-trimethylammonium bromide, cetyl pyridinium chloride, N-myristoyl, trimethyl ammonium chloride).

Personal care products including but not limited to deodorants, toilet soap, and skin lotions containing compounds according to the invention are comprised from formulations readily available in the literature, (Appell 1982). Underarm deodorants and antiperspirants containing compounds according to the invention may comprise a gel, roll-on or stick configuration.

Home care products including but not limited to air fresheners, carpet fresheners and surface cleaners containing compounds according to the invention may comprise anionic and nonionic surfactants, germicides, inert carriers, and fragrance. More specifically, room and air fresheners containing compounds according to the invention may be constituted in the form of liquids, solids, gels, or aerosol formulations. Carpet fresheners containing compounds according to the invention may be constituted in the form of powders; and surface cleaners containing compounds according to the invention may be constituted in the form of powders, liquid concentrates, and liquid sprays.

Fabric care products including but not limited to laundry detergents, fabric conditioners, and fabric fresheners containing compounds according to the invention may also include anionic and nonionic surfactants, cationic softeners, inert carriers and fragrance.

In order to illustrate the invention the following examples are provided. These examples are intended to be exemplary and not exhaustive of the scope of the invention.

### EXAMPLES

### Example 1

2-Isobutyl-[1,3]dioxane:

Isovaleraldehyde (1 mmol) and 1,3-propanediol (1 mmol) were mixed with macro porous polystyrene supported sulfonic acid (60 mg, 0.096 mmol) and anhydrous Na₂SO₄ (700 mg) in CH₂Cl₂ (2 mL). The reaction mixture was shaken overnight, then filtered and concentrated to give 98% pure 2-Isobutyl-[1,3]dioxane, according to GC-MS, in 95% yield.

### Example 2

2-(2-Methyl-cyclopropyl)-[1,3]dioxolane:

2-Methylcyclopropanemethanol was oxidized to intermediate 2-methylcyclopropanecarboxaldehyde under standard oxidation conditions using PDC. The reaction mixture was filtered through Celite and concentrated to give crude aldehyde intermediate, which was reacted with ethylene glycol in the presence of solid supported sulfonic acid and anhydrous Na₂SO₄ in CH₂Cl₂ overnight. After filtration, the reaction mixture was washed with aqueous sodium bicarbonate, water, and brine. Solvent was evaporated under reduced pressure and the residue distilled to give pure product, b.p. 90° C./380 mmHg.

### Example 3

3-Methyl-1,1-dipropoxybutane:

n-Propyl alcohol (0.87 mL, 12.0 mmol, 2.4 equiv) and isovaleraldehyde (0.54 mL, 5.0 mmol) were added to a suspension of resin bound PTS (70-90 mesh; 1.60 mmol/g; 0.25 mmol; 156 mg) and anhydrous Na₂SO₄ (2 g) in CH₂Cl₂ (10 mL). The mixture was shaken vigorously for 40 h, then filtered and concentrated.

### Example 4

1,1-Dimethoxy-3-methylbutane:

A catalytic amount of neat acetyl chloride (1.0 mL) was added slowly to a solution of isovaleraldehyde (10 mL, 93 mmol) in methanol (30 mL) and the reaction mixture allowed to stir overnight at rt. Solid K₂CO₃ was added to quench the reaction and the methanol was carefully evaporated. The crude residue was distilled from fresh K₂CO₃ to afford a pure fraction of the desired acetal (1.7 mL).

### Example 5

6-Isobutyl-5,7-dioxaspiro[2.5]octane:

Isovaleraldehyde (0.54 g, 5.0 mmol) and (1-Hydroxymethyl-cyclopropyl)-methanol (0.51 g, 5.0 mmol) were added to a suspension of resin bound PTS (70-90 mesh; 1.60 mmol/g; 0.25 mmol; 156 mg) and anhydrous Na₂SO₄ (2 g) in CH₂Cl₂ (10 mL). The mixture was shaken vigorously overnight at rt, then filtered and concentrated.

### Example 6

Acetal Matrix Library.

A list of small aldehydes and diols were collected, and the aldehydes were separated in sub-groups of 4 each and diols sub-groups of 6 each. Each aldehyde sub-group was reacted with any one of the diol sub-groups in a 24-well microtiter plate in Matrix format. Thus, in each well, 500 μL solution of 1 M aldehydes in CH₂Cl₂ was mixed with 550 μL of 1 M diols in THF, followed by the addition of the mixture of solid supported sulfonic acid (˜30 mg) and sodium sulfate (˜350 mg). The plates were sealed and shaken at room temperature or 40° C. overnight. In a new 24-well filter plate, 4 mL hydromatrix was loaded to every well. The hydromatrix was treated with 1.0 mL 0.5 N aq. NaOH for 3 min. The cooled reaction mixtures were transferred from the reactor to the filter plate and washed with 4 ml CH₂Cl₂/well. The filtrates were drained and collected in a new 24-well plate. Solvents were evaporated to give products, which were analyzed by GC-MS.

### Example 7

2-(2-Methylcyclopropane)ethanol:

To a solution of diethylzinc (40 mmol, 4 equiv) in DCM-hexanes-DME at −10° C. under argon, solution of CH₂₁₂/DCM (35 mmol, 3.5 equiv) was added over 30 min. Then solution of 2-penten-1-ol (10 mmol) in DCM was added drop wise, the reaction mixture was shaken for 1 h at −10° C., and overnight at room temperature. The mixture was quenched by saturated aqueous ammonium chloride solution and extracted using ether. The organic layers were combined and washed sequentially with 10% aq. HCl, saturated sodium bicarbonate, saturated Na₂S₂O₃ twice, water and brine. After evaporating solvent, the crude residues were subjected to vacuum distillation to afford pure product.

### Example 8

3-Methylbutyric acid propyl ester:

Isovaleric acid chloride (50 mmol) was allowed to react with n-propyl alcohol (50 mmol) in the presence of triethylamine (60 mmol) in CH₂Cl₂ overnight at rt. The reaction mixture was washed with water, saturated sodium bicarbonate, water, and brine. The organic phase was dried over anhydrous Na₂SO₄ and solvent removed to afford the desired ester.

### Example 9

Blocking the Perception of Isovaleric Acid by Low Molecular Weight Acetals, Esters and Alcohols.

a) Malodor Reduction Test

Each subject's sense of smell was tested using a smell identification test and an isovaleric acid threshold test. To pass the smell ID test the subject had to correctly identify the aromas of peach, spearmint, cinnamon, lime, pineapple and clove. To pass the isovaleric acid threshold test subjects had to detect the odor of isovaleric acid in a one part-per-million (1 ppm weight/volume) aqueous isovaleric acid solution. This is above the 0.12 ppm threshold for a person with a normal sense of smell and below the 5.0 ppm threshold for a person with a specific anosmia to isovaleric acid (Amoore 1977). The use of an odor ID and a threshold test to identify subjects with a normal sense of smell is standard practice, (Cain, Gent et al. 1983).

The target malodor, isovaleric acid, was presented to the subject in a 60 mL, wide mouth amber, screw cap jar. The concentration of isovaleric acid in water that gave an odor intensity of strong or higher on the Labeled Magnitude Scale was 56 parts-per-million (weight/volume). Five milliliters of a 1% (v/v) solution of the test substance in diethylphthalate (DEP) presented in a 20 ml glass vial were used to evaluate the blocker candidate. The labeled the Labeled Magnitude Scale (LMS, FIG. 1) was developed for the quantitative measurement of taste, irritation and odor intensities (Green, Shaffer et al. 1993; Green, Dalton et al. 1996).

Evaluation of a sample for malodor blocking was as follows: a) The subject sniffed the isovaleric acid (target) with both nostrils and scored its intensity using the Labeled Magnitude Scale (LMS), b) The subject waited one minute, c) the subject sniffed the candidate compound twice with the left and then twice with the right nostril, d) the subject sniffed the isovaleric acid target sample within 20 seconds with both nostrils and scored its odor using the LMS, and e) the subject waited for two minutes before proceeding to the next test substance.

The percent malodor reduction (% MOR) was calculated using the following formula:

% MOR=[1−(IVAInt after CA/IVAINT before CA)]×100

where: IVA isovaleric acid

INT =intensity

The ratio in this equation goes from zero (complete blocking) to one (no blocking). The % malodor reduction moves in the opposite direction and goes from 100% for complete blocking to 0% for no blocking.

B. Results

Tables 1, 2, and 3 present the percent malodor reduction for some low molecular weight acetals, alcohols and esters respectively. In each of these tables the standard error of the mean is the standard deviation of the mean divided by the square root of the number of subjects used for the evaluation.

TABLE 1
Percent Malodor Reduction for Some Low Molecular Weight Acetals

| | | Malodor | Standard |
|---|---|---|---|
| Compound Name | Structure | Reduction (%) | Error |
| 2-Isobutyl-[1,3]dioxane | 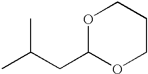 | 97 | 1 |
| 2-Isobutyl-4-methyl- [1,3]dioxane | 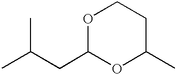 | 92 | 3 |
| 2-Isobutyl-4,5-dimethyl- [1,3]dioxolane | 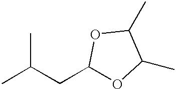 | 89 | 4 |
| 2-Isobutyl-4-methyl- [1,3]dioxolane | 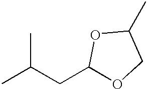 | 89 | 5 |
| 1,1-Dimethoxy-3-methyl- butane | 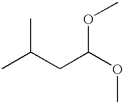 | 87 | 6 |
| 2-Isobutyl-[1,3]dioxolane | 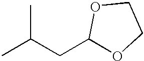 | 87 | 2 |
| 2-(2-Methyl-cyclopropyl)- [1,3]dioxolane | 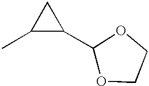 | 85 | 5 |
| 2-Isobutyl-5-methyl- [1,3]dioxane | 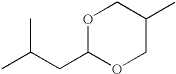 | 80 | 7 |
| 1,1-Diethoxy-3-methyl- butane | 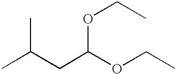 | 78 | 12 |
| 4-Ethyl-2-isobutyl- [1,3]dioxolane | 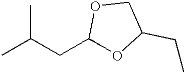 | 78 | 6 |

TABLE 2
Percent Malodor Reduction for Some Low Molecular Weight Alcohols

| | | Malodor | Standard |
|---|---|---|---|
| Compound Name | Structure | Reduction (%) | Error |
| 2-Methylcyclopropane methanol | 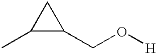 | 85 | 5 |
| 2-(2-Methyl-cyclopropyl)- ethanol | 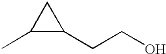 | 77 | 7 |
| 2-Cyclopropylethanol | 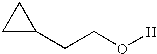 | 74 | 6 |
| Isoamyl Alcohol | 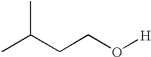 | 73 | 10 |
| 2-(1-Methyl-cyclopropyl)- ethanol | 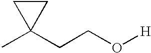 | 72 | 10 |

TABLE 3
Percent Malodor Reduction for Some Low Molecular Weight Esters.

| | | Malodor | Standard |
|---|---|---|---|
| Compound Name | Structure | Reduction (%) | Error |
| Ethyl isovalerate | 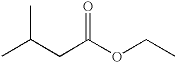 | 94 | 7 |
| Methyl isovalerate | 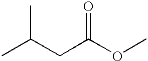 | 92 | 4 |
| 2-Methyl- cyclopropanecarboxylic acid methyl ester | 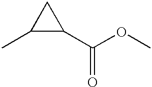 | 89 | 5 |
| n-Propyl isovalerate | 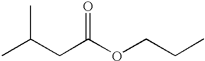 | 83 | 3 |
| 2-Methyl- cyclopropanecarboxylic acid ethyl ester | 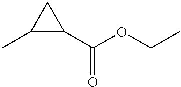 | 81 | 7 |
| Isopropyl isovalerate | 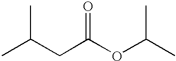 | 74 | 11 |
| 2-Methyl- cyclopropanecarboxylic acid isopropyl ester | 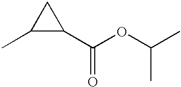 | 68 | 8 |
| Isoamylacetate | 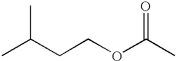 | 67 | 10 |

### Example 10

A conventional deodorant stick composition, for example one with the following composition:

| | |
|---|---|
| 65.00 | PROPYLENE GLYCOL |
| 27.00 | DISTILLED OR DEIONIZED WATER |
| 7.00 | SODIUM STEARATE C-1 (WITCO) |
| 1.00 | FRAGRANCE |
| 100.00 | |

is modified by the addition of compound having a malodor reduction value of 80% or more as measured by the malodor reduction test presented in Example 1 in an amount such that it accounts for 1% by weight of the deodorant stick. The deodorant stick containing 1% of the malodor reduction compound was found to completely block the perception isovaleric acid when the stick was sniffed twice before sniffing an aqueous solution containing 56 parts-per million of isovaleric acid. The deodorant stick containing fragrance alone did not block the perception of isovaleric acid. Examples of effective compounds are: 2-Isobutyl-[1,3]dioxane, 2-Isobutyl-4,5-dimethyl-[1,3]dioxolane, 2-Isobutyl-4-methyl-[1,3]dioxane, 2-Isobutyl-4-methyl-[1,3]dioxolane, 1,1-Dimethoxy-3-methyl-butane, 2-lsobutyl-[1,3]dioxolane, 2-(2-Methyl-cyclopropyl)-[1,3]dioxolane, 2-Isobutyl-5-methyl-[1,3]dioxane, 2-Methylcyclopropane methanol, Ethyl isovalerate, Methyl isovalerate, 2-Methyl-cyclopropanecarboxylic acid methyl ester, n-Propyl isovalerate, and 2-Methyl-cyclopropanecarboxylic acid ethyl ester.

While the invention has been described by way of specific embodiments, it is understood that the words have been used herein are words of description, rather than words of limitation. Changes may be made, within the purview of the appended claims, without departing from the scope and spirit of the invention in its broader aspects. Although the invention has been described herein with reference to particular means, materials and embodiments, it is understand that the invention is not limited to the particular disclosed. The invention extends to all equivalent structures, means and uses which are within the scope of the appended claims.

### REFERENCES

Amoore, J. E. (1977). “Specific anosmia and the concept of primary odors.”Chem. Senses Flavor 2(3): 267-81.

Appell, L. (1982). Cosmetics, Fragrances and Flavors. Whiting, N.J. 08759, Novox Inc.

Cain, W. S. (1975). “Odor Intensity: Mixtures and Masking.” Chem. Senses 1: 339-352.

Cain, W. S., Drexler, W. (1974). “Scope and Evaluation of Odor Counteraction and Masking.” Annals of the New York Academy of Sciences 237: 427-439.

Cain, W. S., J. Gent, et al. (1983). “Clinical evaluation of olfaction.” AMERICAN JOURNAL OF OTOLARYNGOLOGY 4(4): 252-6.

Cain, W. S. and E. H. Polack (1992). “Olfactory adaptation as an aspect of odor similarity.” Chemical Senses 17: 481-491.

Green, B. G., P. Dalton, et al. (1996). “Evaluating the ‘Labeled Magnitude Scale’ for measuring sensations of taste and smell.” CHEMICAL SENSES 21(3): 323-34:.

Green, B. G., G. S. Shaffer, et al. (1993). “Derivation and evaluation of a semantic scale of oral sensation magnitude and apparent ratio properties.” CHEMICAL SENSES 18: 683-702.

Labows, J. N., J. T. Reilly, et al. (1999). “Axillary odor determination, formation, and control.” Cosmet. Sci. Technol. Ser. 20(Antiperspirants and Deodorants (2nd Edition)): 59-82.

Marano, F. A., J. T. Van Elst, et al. (2001). Human sweat malodor counteractant composition and process for using same. Eur. Pat. Appl. 1113105. Ep, (International Flavors & Fragrances Inc., USA).: 21 pp.

Pierce, J. D., Jr., D. H. Blank, et al. (1996). “Cross-adaptation of sweaty-smelling 3-methyl-2-hexenoic acid by its ethyl esters is determined by structural similarity.” J. Soc. Cosmet. Chem. 47(6): 363-375.

Pierce, J. D., Jr., D. H. Blank, et al. (1998). “Reduction in the sweaty smell of 3-methyl-2-hexenoic acid by cross-adaptation using its pleasant-smelling methyl esters.” J. Cosmet. Sci. 49(6): 369-376.

Preti, G., J. J. D. Pierce, et al. (1996). Method for reducing perception of human underarm odor by a pleasant smelling compound. U.S. Pat. No. 5,538,719, (Monell Chemical Senses Center, USA).: 14 pp. Cont.-in-part of U.S. Ser. No. 67,672.

1. A method for blocking or reducing the odor of a malodorous carboxylic acid selected from the group consisting of butyric acid, propionic acid, isovaleric acid, hexanoic acid and 3-methyl-2-hexenoic acid comprising contacting an environment or substrate prone to accumulation or contamination of one or more of said malodorous carboxylic acids with an odor reducing effective amount of an acetal compound having the following structure: 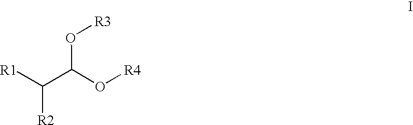
wherein R1 is selected from the group consisting of 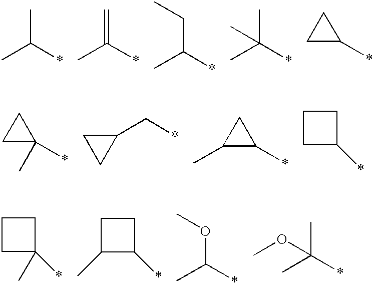
R2 is selected from the group consisting of H, Me, Et, OH, and OMe;
R3 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂,CH₃, —CH₂CH₂,CH₂CH₃;
R4 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂CH₃, —CH₂CH₂OCH₂CH₃;

2. The method of claim 1 wherein said compound is selected from the group consisting of

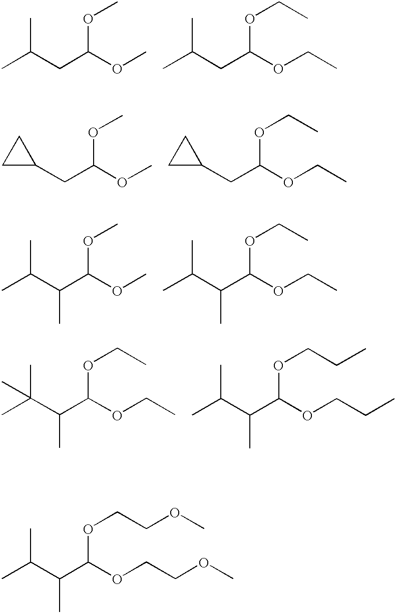

3. The method of claim 1 wherein the substrate is selected from the group consisting of human or animal skin, air, a fabric, and a carpet or rug.

4. The method of claim 1 wherein the malodorous carboxylic acid is isovaleric acid.

5. The method of claim 1 wherein the malodorous carboxylic acid is 3-methyl-2-hexenoic acid.

6. A method for blocking or reducing the odor of a malodorous carboxylic acid selected from the group consisting of butyric acid, isovaleric acid, propionic acid, hexanoic acid and 3-methyl-2-hexenoic acid comprising contacting an environment or substrate prone to accumulation or contamination with one or more of said malodorous carboxylic acids with a substituted 1,3-dioxacycle compound having one of the following structures: 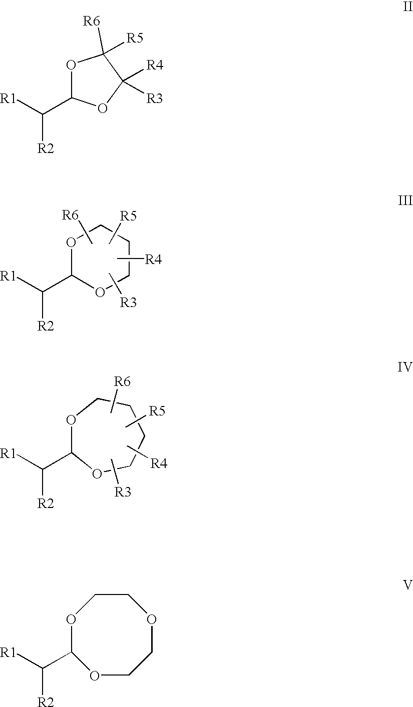
wherein, R1 is selected from 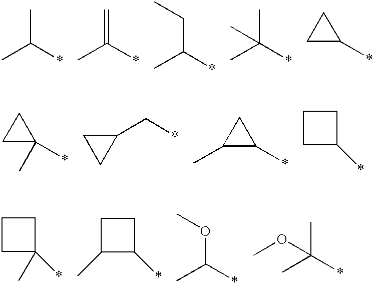
R2 is selected from the group consisting of H, Me, Et, OH and OMe;
R3 represents unsubstitution or is selected from small normal and branched alkanes and alkenes, and cycloalkyl and heteratom substituted alkanes and alkenes.
R4 represents unsubstitution or is selected from small normal and branched alkanes and alkenes, and cycloalkyl and heteratom substituted alkanes and alkenes.
R5 represents unsubstitution or is selected from small normal and branched alkanes and alkenes, and cycloalkyl and heteratom substituted alkanes and alkenes.
R6 represents unsubstitution or is selected from small normal and branched alkanes and alkenes, and cycloalkyl; and heteratom substituted alkanes and alkenes.
R3 and R4 can be combined to represent carbonyl (═O), or olefin (C═CH₂), or spirocyclic derivatives.
R4 and R5 can be combined to form fused cyclic derivatives.

7. The method of claim 6 wherein said substituted 1 ,3-dioxacycle is selected from the following structures:

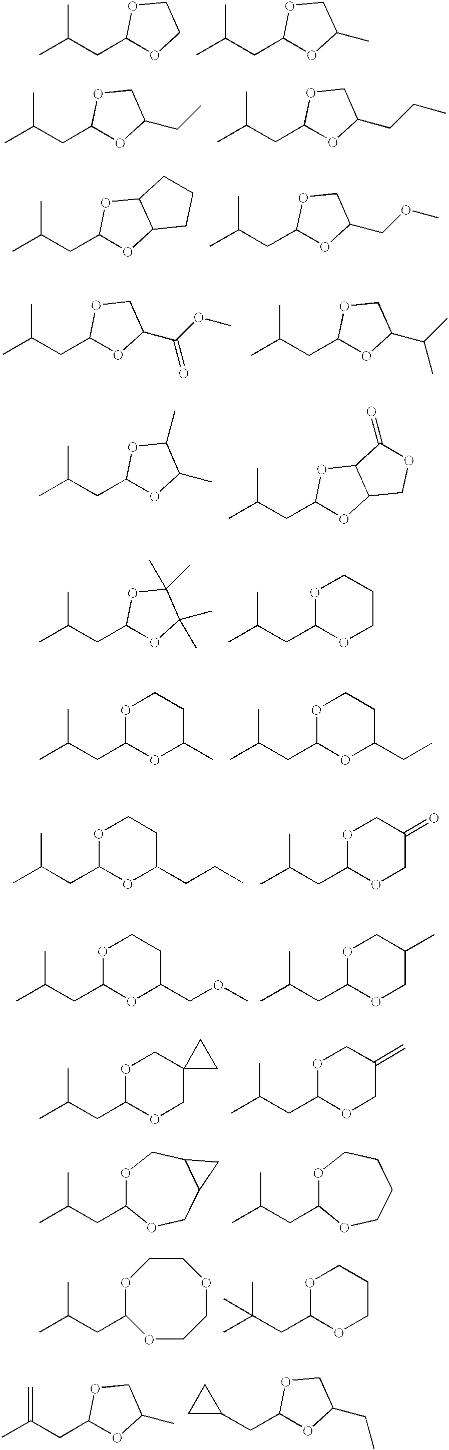

8. A method for blocking or reducing the odor of a malodorous carboxylic acid selected from the group consisting of butyric acid, isovaleric acid, propionic acid, hexanoic acid and 3-methyl-2-hexenoic acid comprising contacting an environment or substrate prone to accumulation or contamination with one or more of said malodorous carboxylic acids with a substituted cyclopropyl or cyclobutyl-substituted 1,3-dioxacyclic compound having one of the following structures: 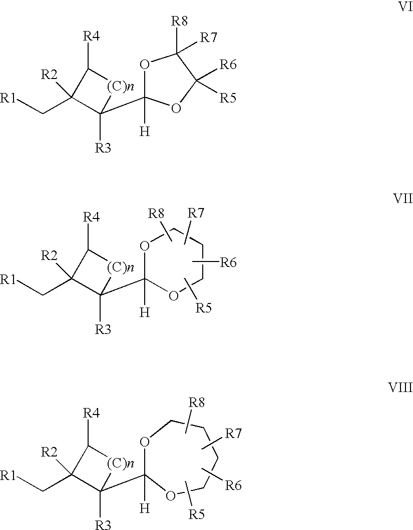
wherein R1 is selected from a group consisting of H, Me and methylene (═CH₂);
R2 is H, or methyl;
R3 is H, methyl, or methoxy (OMe);
R4 is H or methyl;
n is 0 or 1;
R5 represents unsubstitution, or is selected from small normal and branched alkanes and alkenes, and heteratom substituted alkanes and alkenes,
R6 represents unsubstitution, or is selected from small normal and branched alkanes and alkenes, and heteratom substituted alkanes and alkenes,
R7 represents unsubstitution or is selected from small normal and branched alkanes and alkenes, and heteratom substituted alkanes and alkenes,
R8 represents unsubstitution , or is selected from small normal and branched alkanes and alkenes and heteratom substituted alkanes and alkenes or
R5 and R6 together represent carbonyl (═O), or olefin (C═CH₂), or spirocyclic derivatives. and
R6 and R7 are combined to represent fused cyclic derivatives.

9. The method of claim 8 wherein said cyclopropyl or dioxacycle compound is selected from the group consisting of:

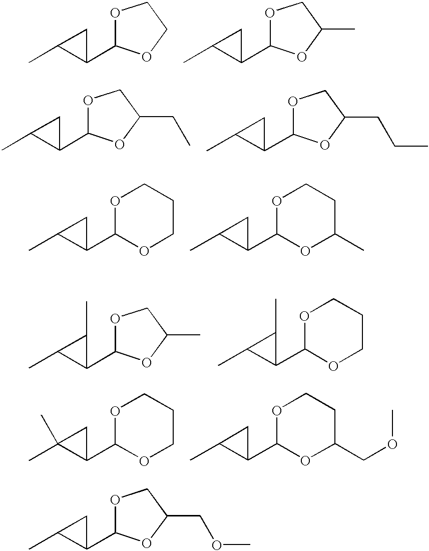

10. A method for blocking or reducing the odor of a malodorous carboxylic acid selected from the group consisting of butyric acid, isovaleric acid, propionic acid, hexanoic acid, and 3-methyl-2-hexenoic acid comprising contacting an environment or substrate prone to accumulation or contamination with one or more of said malodorous carboxylic acids with a substituted cyclopropyl or cyclobutyl-substituted O-alkyl acetal compound having the following generic structure: 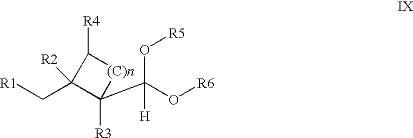
wherein R1 is selected from a group consisting of H, Me and methylene (═CH₂);
R2 is H, or methyl;
R3 is H, methyl, or methoxy (OMe);
R4 is H or methyl;
n is 0 or 1;
R5 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂OCH₃, and —CH₂CH₂OCH₂CH₃; and
R6 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂OCH₃, and —CH₂CH₂OCH₂CH₃.

11. The method of claim 10 wherein said compound is selected from the group consisting of

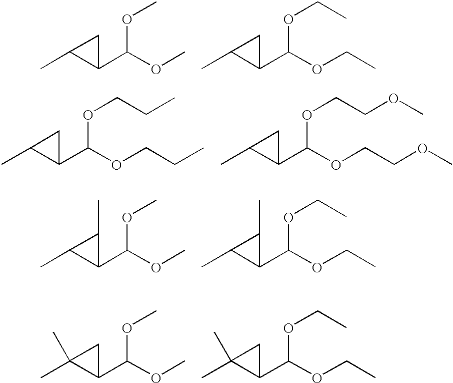

12. A method for blocking or reducing the odor of a malodorous carboxylic acid selected from the group consisting of butyric acid, isovaleric acid, 3-methyl-2-hexenoic acid, hexanoic acid and propionic acid comprising contacting an environment or substrate prone to accumulation or contamination by one or more of said carboxylic acids with an effective, odor blocking or reducing amount of at least one compound selected from the group consisting of: a) 2-Isobutyl-[1,3]dioxane, b) 2-Isobutyl-4-methyl-[1,3]dioxane, c) 2-Isobutyl-4,5-dimethyl-[1,3]dioxolane, d) 2-Isobutyl-4-methyl-[1,3]dioxolane, e) 1,1-Dimethoxy-3-methyl-butane, f ) 2-lsobutyl-[1,3]dioxolane, g) 2-(2-Methyl-cyclopropyl)-[1,3]dioxolane, h) 2-Isobutyl-5-methyl-[1,3]dioxane, 1) 1,1-Diethoxy-3-methyl-butane, j) 4-Ethyl-2-isobutyl-[1,3]dioxolane, k) 2-Isobutyl-4-isopropyl-[1,3]dioxolane, l) 2-Isobutyl-4-propyl-[1,3]dioxolane, m) 2-Isobutyl-5-methylene-[1,3]dioxane, n) 2-Isobutyl-tetrahydro-cyclopenta[1,3]dioxole, o) 2-Isobutyl-4,4,5,5-tetramethyl-[1,3]dioxolane and p) 2-Isobutyl-4-methoxymethyl-[1,3]dioxolane.

13. The method of claim 12 wherein said compound is 2-isobutyl-[1,3] dioxane.

14. The method of claim 12 wherein said compound is 2-isobutyl-4-methyl-[1,3] dioxane.

15. The method of claim 12 wherein said compound is 2-Isobutyl-4,5-dimethyl-[1,3]dioxolane

16. The method of claim 12 wherein said compound is 2-Isobutyl-4-methyl-[1,3]dioxolane.

17. The method of claim 12 wherein said compound is 2-isobutyl-[1,3]dioxolane.

18. The method of claim 12 wherein said compound is 2-Isobutyl-5-methyl-[1,3]dioxane.

19. The method of claim 12 wherein said compound is 4-Ethyl-2-isobutyl-[1,3]dioxolane.

20. The method of claim 12 wherein said compound is 2-Isobutyl-4-isopropyl-[1,3]dioxolane.

21. The method of claim 12 wherein said compound is 2-Isobutyl-4-propyl-[1,3]dioxolane.

22. The method of claim 12 wherein said compound is 1,1-Dimethoxy-3-methyl-butane.

23. The method of claim 12 wherein said compound is 1,1-Diethoxy-3-methyl-butane.

24. The method of claim 12 wherein said compound is and 2-Isobutyl-4-methoxymethyl-[1,3] dioxolane.

25. The method of claim 12 wherein a composition containing said compound is topically applied to a human or animal subject.

26. The method of claim 12 wherein a composition containing said compound is applied to a substrate selected from clothing, a rug or carpet, or upholstery.

27. The method of claim 12 wherein a composition containing said compound is sprayed in an aerosol formulation.

28. A composition adapted for blocking or reducing the odor of at least one carboxylic acid selected from the group consisting of isovaleric acid, butyric acid, 3-methyl-2-hexenoric acid, hexenoic acid and propionic acid comprising an effective odor blocking or reducing effective amount of at least one O-alkyl substituted acetal compound having the following structure: 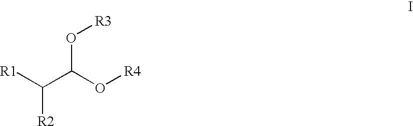
wherein R1 is selected from the group consisting of 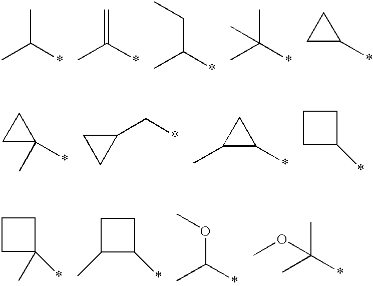
R2 is selected from the group consisting of H, Me, Et, OH, and OMe;
R3 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂OCH₃, and —CH₂CH₂OCH₂CH₃; and
R4 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂OCH₃, and —CH₂CH₂OCH₂CH₃.

29. The composition of claim 28 wherein said acetal compound is selected from the group consisting of

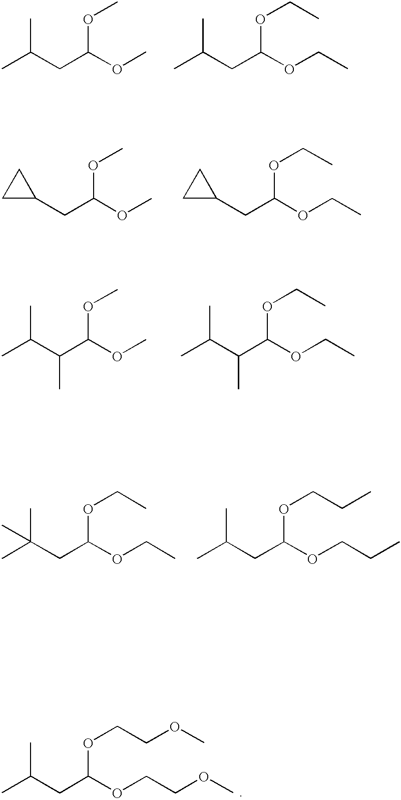

30. The composition of claim 28 which is selected from the group consisting of: a) personal care products including underarm deodorants and antiperspirants, hair shampoos and conditioners, toilet soaps, and skin lotions; b) fabric care products including laundry detergents, fabric conditioners, and fabric fresheners; c) pet care products including deodorants, shampoos, conditioners: and d) home care products including air fresheners, carpet fresheners, surface cleaners and hand wash dish detergents.

31. A composition adopted for blocking or reducing the odor of at least one carboxylic acid from the group consisting of isovaleric acid, butyric acid, 3-methyl-2-hexanoic acid, hexanoic acid, and propionic acid comprising an effective odor-reducing amount of at least one substituted 1,3-dioxacycle compound having one of the following generic structures: 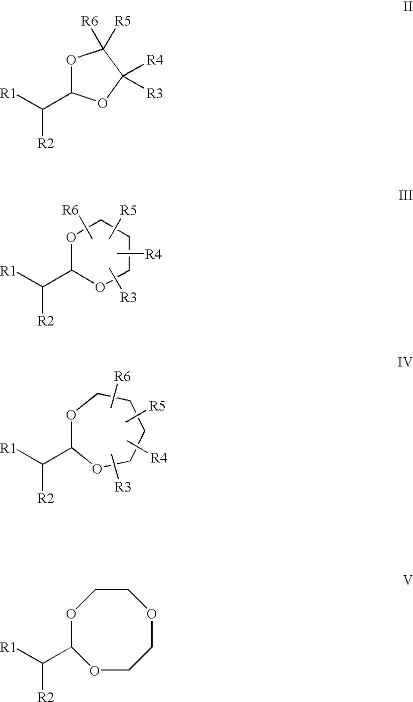
wherein, R1 is selected from 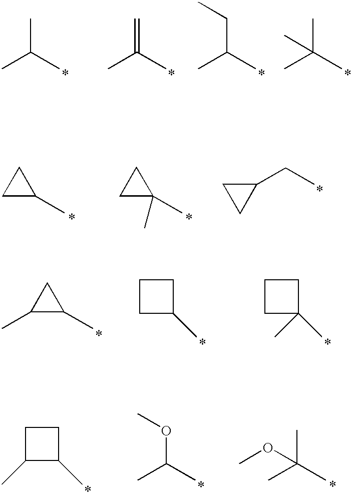
R2 is selected from the group consisting of H, Me, Et, OH and OMe;
R3 represents unsubstitution, or is selected from small normal and branched alkanes and alkenes, and cycloalkyl and heteratom substituted alkanes and alkenes,
R4 represents unsubstitution, or is selected from small normal and branched alkanes and alkenes, and cycloalkyl and heteratom substituted alkanes and alkenes,
R5 represents unsubstitution, or is selected from small normal and branched alkanes and alkenes, and cycloalkyl and heteratom substituted alkanes and alkenes.
R6 represents unsubstitution or is selected from small normal and branched alkanes and alkenes, and cycloalkyl and heteratom substituted alkanes and alkenes.
R3 and R4 can be combined to represent carbonyl (═O), or olefin (C═CH₂), or spirocyclic derivatives; or
R4 and R5 are combined to represent fused cyclic derivatives.

32. The composition of claim 31 wherein said compound is selected from the group consisting of

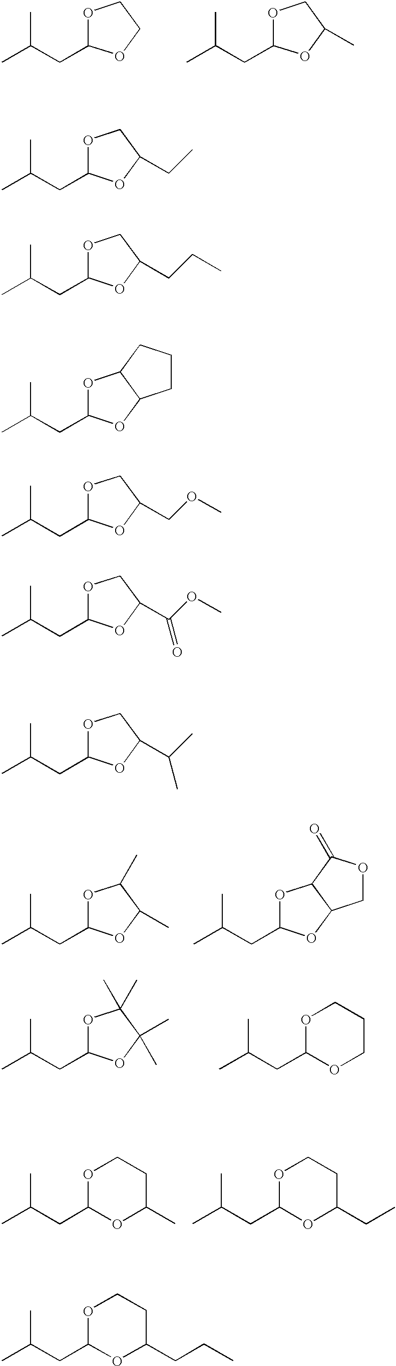

33. The composition of claim 31 which is selected from the group consisting of : a) personal care products including underarm deodorants and antiperspirants, hair shampoos and conditioners, toilet soaps, and skin lotion; b) fabric care products including laundry detergents, fabric conditioners, and fabric fresheners; c) pet care products including deodorants, shampoos, conditioners: and d) home care products including air fresheners, carpet fresheners, surface cleaners and hand wash dish detergents.

34. A composition adapted for blocking or reducing the odor of at least one carboxylic acid selected from the group consisting of isovaleric acid, butyric acid, 3-methyl-2-hexenoic acid, hexanoic acid, and propionic acid comprising an effective odor reducing effective amount of at least one substituted cyclopropyl or cyclobutyl-substituted 1,3-dioxacyclo compound having a generic structure VI, VII or VIII as set forth below: 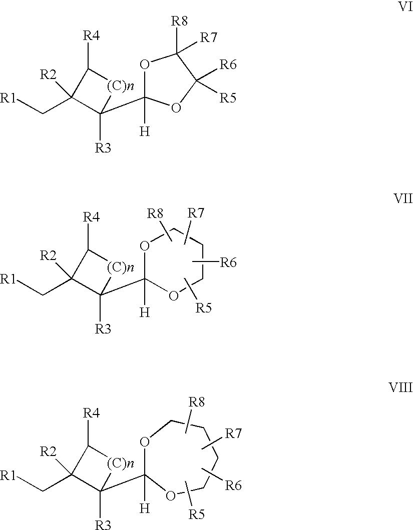
wherein R1 is selected from a group consisting of H, Me and methylene (═CH₂);
R2 is H, or methyl;
R3 is H, methyl, or methoxy (OMe);
R4 is H or methyl;
n is 0 or 1;
R5 represents unsubstitution or is selected from small normal and branched alkanes and alkenes, and cycloalkyl; and heteratom substituted alkanes and alkenes.
R6 represents unsubstitution or is selected from small normal and branched alkanes and alkenes, and cycloalkyl and heteratom substituted alkanes and alkenes.
R7 represents unsubstitution or is selected from small normal and branched alkanes and alkenes, and cycloalkyl and heteratom substituted alkanes and alkenes.
R8 represents unsubstitution or is selected from small normal and branched alkanes and alkenes, and cycloalkyl and heteratom substituted alkanes and alkenes. or
R5 and R6 are combined to represent carbonyl (═O), or olefin (C═CH₂), or spirocyclic derivatives. and
R6 and R7 can be combined to represent fused cyclic derivatives wherein R3 is selected from the group consisting of H, Me, Et, OH and OMe.
R5 is H, a C1-C10 alkyl or a cycloalkyl selected from the group consisting of cyclobutyl, cyclopropyl, cyclopentyl, cyclohexyl and cyclooctyl;
R6 is selected from the group consisting of H, Me, and CH₂═;
R7 is H or Me;
R8 is H or Me; and
n is 0 or 1.

35. The composition of claim 34 wherein said dioxacyclo compound is selected from the group consisting of

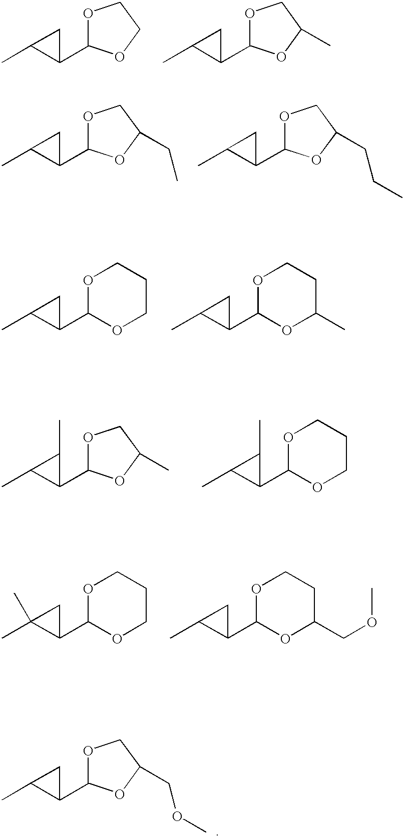

36. The composition of claim 34 which is selected from the group consisting of: a) personal care products including underarm deodorants and antiperspirants, hair shampoos and conditioners, toilet soaps, and skin lotions; b) fabric care products including laundry detergents, fabric conditioners, and fabric fresheners; c) pet care products including deodorants, shampoos, conditioners: and d) home care products including air fresheners, carpet fresheners, surface cleaners and hand wash dish detergents.

37. A composition adapted for blocking or reducing the odor of at least one carboxylic acid selected from the group consisting of isovaleric acid, butyric acid, 3-methyl-2-hexenoic acid, hexanoic acid and propionic acid comprising an effective odor reducing amount of at least one substituted cyclopropyl or cyclobutyl-substituted O-alkyl acetal having generic formula IX below: 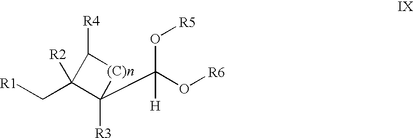
wherein R1 is selected from a group consisting of H, Me and methylene (═CH₂);
R2 is H, or methyl;
R3 is H, methyl, or methoxy (OMe);
R4 is H or methyl;
n is 0 or 1;
R5 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂OCH₃, —CH₂CH₂OCH₂CH₃;
R6 is selected from the group consisting of Me, Et, Pr, iPr, Bu, and —CH₂cyclopropyl, —CH₂CH═CH₂, —CH₂CH═CHCH₃, —CH₂CH₂CH═CH₂, —CH₂CH₂OCH₃, —CH₂CH₂OCH₂CH₃.

38. The composition of claim 37 wherein said compound is selected from the group consisting of

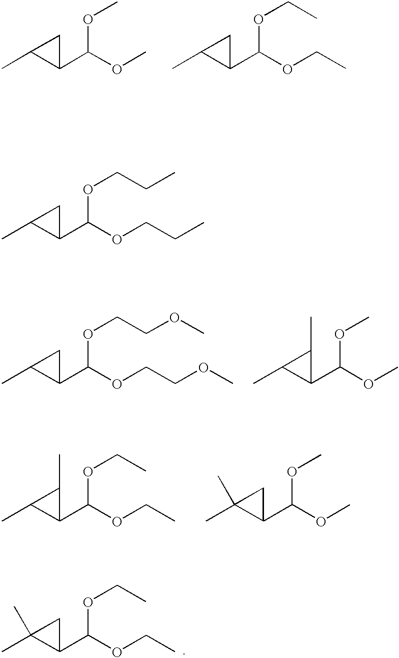

39. The composition of claim 37 which is selected from the group consisting of: a) personal care products including underarm deodorant and antiperspirant, hair shampoo and conditioner, toilet soap, and skin lotion; b) fabric care products including laundry detergents, fabric conditioners, and fabric fresheners; c) pet care products including deodorant, shampoo, conditioner: and d) home care products including air fresheners, carpet fresheners, surface cleaners and hand wash dish detergent.

40. A composition adapted for blocking or reducing the odor of at least one carboxylic acid selected from the group consisting of isovaleric acid, butyric acid, 3-methyl-2-hexenoic acid and propionic acid comprising an effective, odor blocking or reducing effective amount of at least one acetal compound selected from the group consisting of: a) 2-Isobutyl-[1,3]dioxane, b) 2-Isobutyl-4-methyl-[1,3]dioxane, c) 2-Isobutyl-4,5-dimethyl-[1,3]dioxolane, d) 2-Isobutyl-4-methyl-[1,3]dioxolane, e) 1,1-Dimethoxy-3-methyl-butane, f ) 2-lsobutyl-[1,3]dioxolane, g) 2-(2-Methyl-cyclopropyl)-[1,3]dioxolane, h) 2-Isobutyl-5-methyl-[1,3]dioxane, 1) 1,1-Diethoxy-3-methyl-butane, j) 4-Ethyl-2-isobutyl-[1,3]dioxolane, k) 2-Isobutyl-4-isopropyl-[1,3]dioxolane, l) 2-Isobutyl-4-propyl-[1,3]dioxolane, m) 2-Isobutyl-5-methylene-[1,3]dioxane, n) 2-Isobutyl-tetrahydro-cyclopenta[1,3]dioxole, o) 2-Isobutyl-4,4,5,5-tetramethyl-[1,3]dioxolane, and p) 2-Isobutyl-4-methoxymethyl-[1,3]dioxolane.

41. The composition of claim 40 wherein said compound is 2-isobutyl-[1,3] dioxane.

42. The composition of claim 40 wherein said compound is 2-isobutyl-4-methyl-[1,3]dioxane.

43. The composition of claim 40 wherein said compound is 2-isobutyl-4,5-dimethyl-[1,3]dioxolane.

44. The composition of claim 40 wherein said compound is 2-isobutyl-4-methyl-[1,3]dioxolane.

45. The composition of claim 40 wherein said compound is 1,1-dimethoxy-3-methyl-butane.

46. The composition of claim 40 wherein said compound is 2-Isobutyl-[1,3]dioxolane.

47. The composition of claim 40 wherein said compound is 2-Isobutyl-5-methyl-[1,3]dioxane.

48. The composition of claim 40 wherein said compound is 4-Ethyl-2-isobutyl-[1,3]dioxolane.

49. The composition of claim 40 wherein said compound is 2-Isobutyl-4-isopropyl-[1,3]dioxolane.

50. The composition of claim 40 wherein said compound is 2-Isobutyl-4-propyl-[1,3]dioxolane.

51. The method of claim 40 wherein said compound is 1,1-Dimethoxy-3-methyl-butane.

52. The method of claim 40 wherein said compound is 1,1-Diethoxy-3-methyl-butane.

53. The method of claim 40 wherein said compound is 2-Isobutyl-4-methoxymethyl-[1,3]dioxolane.

54. A method for blocking or reducing the odor of a malodorous carboxylic acid selected from the group consisting of butyric acid, propionic acid, isovaleric acid, hexanoic acid and 3-methyl-2-hexenoic acid comprising contacting an environment or substrate prone to accumulation or contamination of one or more of said malodorous carboxylic acids with an odor reducing effective amount of an alcohol or O-acylated derivative having the following general structure: 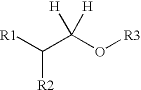
wherein R1 is selected from the group consisting: 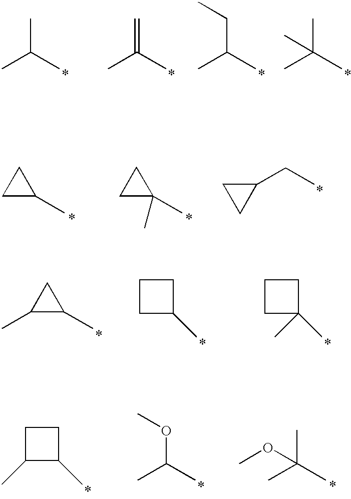
R2 is selected from the group consisting of H, Me, Et, OH, and OMe;
R3 is selected from the group consisting of of H, —C(O)R₄, where R4 is selected from Me, Et, Pr, iPr, Bu, and —CH₂cyclopropyl, —CH₂CH₂CH═CH₂, —CH₂OCH₃, —CH₂COCH₂CH₃, —CH₂CH₂OCH₃, —CH₂CH₂OCH₂CH₃.

55. The method of claim 54 wherein said component is selected from the group consisting of

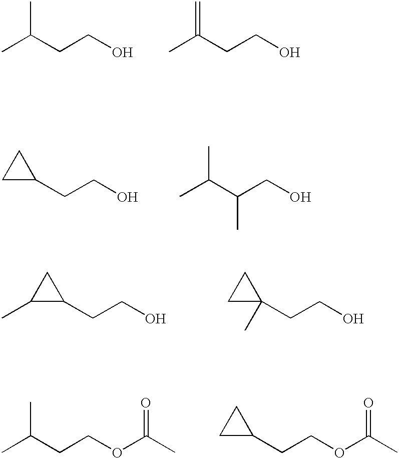

56. The method of claim 54 wherein the environment is selected from the group consisting of human or animal skin, the air, a fabric, and a carpet or rug.

57. The method of claim 54 wherein the malodorous carboxylic acid is isovaleric acid.

58. The method of claim 54 wherein the malodorous carboxylic acid is 3-methyl-2-hexenoic acid.

59. A method for blocking or reducing the odor of a malodorous carboxylic acid selected from the group consisting of isovaleric acid, propionic acid, hexanoic acid and 3-methyl-2-hexenoic acid comprising contacting an environment or substrate prone to accumulation or contamination with one or more of said malodorous carboxylic acids with an alcohol or O-acyl derivative thereof having the following generic structure: 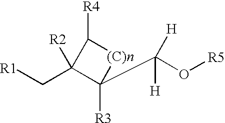
wherein, R1 is H or Me;
R2 is H or Me;
R3 is selected from the group consisting of H, Me, and OMe;
R4 is H or Me;
R5, is selected from the group consisting of H; —C(O)R6, where R6 is selected from the group consisting is selected from Me, Et, Pr, iPr, Bu, and —CH₂cyclopropyl, —CH₂CH₂CH═CH₂, —CH₂OCH₃, —CH₂COCH₂CH₃, —CH₂CH₂OCH₃, and —CH₂CH₂OCH₂CH₃, and n is 0 or 1.

60. The method of claim 59 wherein said substrate is selected from group consisting of

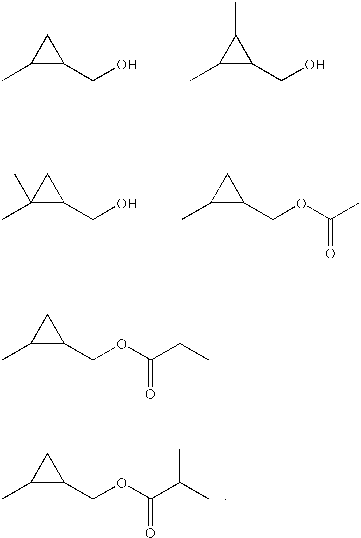

61. The method of claim 59 wherein said substrate is selected from the group consisting of human or animal skin, air, a fabric, and a carpet or rug.

62. The method of claim 59 wherein the malodorous carboxylic acid is isovaleric acid or butyric acid.

63. The method of claim 59 wherein the malodorous carboxylic acid is 3-methyl-2-hexenoic acid.

64. A method for blocking or reducing the odor of a malodorous carboxylic acid selected from the group consisting of butyric acid, isovaleric acid, 3-methyl-2-hexenoic acid, hexanoic acid and propionic acid comprising contacting an environment prone to the accumulation or contamination of one or more malodorous carboxylic acids with an alcohol or O-acylated derivative selected from the group consisting of (2-methylcyclopropyl)methanol, 2-(2-methylcyclopropyl)ethanol, 2-cyclopropyl ethanol, isoamyl alcohol, 2-(1-methylcyclopropyl)ethanol, isoamylacetate, (2,2-dimethylcyclopropyl)methanol and 3-methylbut-3-enol.

65. The method of claim 64 wherein said compound is (2-methylcyclopropyl)methanol.

66. The method of claim 64 wherein said component is 2-(2-methyl-cyclopropyl)ethanol.

67. The method of claim 64 wherein said component is 2-cyclopropyl ethanol.

68. A composition adapted from blocking or reducing the odor of at least one malodorous carboxylic acid selected from the group consisting of isovaleric acid, 3-methyl-2-hexenoic acid, hexanoic acid and propionic acid comprising an effective odor blocking or reducing amount of at least one alcohol or O-acylated derivative having the general structure: 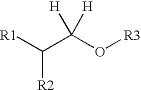
wherein R1 is selected from the group consisting: 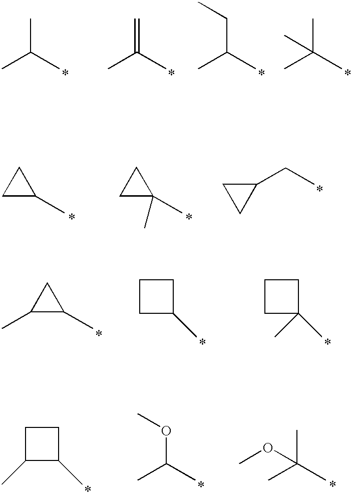
R2 is selected from the group consisting of H, Me, Et, OH, and OMe;
R3 is selected from the group consisting of of H, —C(O)R₄, where R4 is selected from Me, Et, Pr, iPr, Bu, and —CH₂cyclopropyl, —CH₂CH₂CH═CH₂, —CH₂OCH₃, —CH₂COCH₂CH₃, —CH₂CH₂OCH₃, —CH₂CH₂OCH₂CH₃.

69. The composition of claim 68 wherein said component is selected from the group consisting of

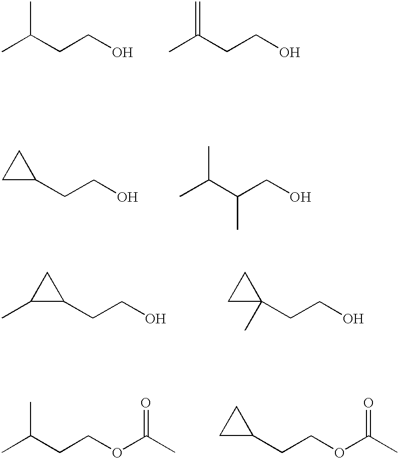

70. The composition of claim 68 which is selected from the group consisting of: a) personal care products including underarm deodorants and antiperspirants, hair shampoos and conditioners, toilet soaps, and skin lotions; b) fabric care products including laundry detergents, fabric conditioners, and fabric fresheners; c) pet care products including deodorants, shampoos, conditioners: and d) home care products including air fresheners, carpet fresheners, surface cleaners and hand wash dish detergents.

71. A composition adapted for blocking or reducing the odor of at least one malodorous carboxylic acid selected from the group consisting of isovaleric acid, 3-methyl-2-hexenoic acid, hexanoic acid and propionic acid comprising an effective odor blocking or reducing amount of at least one alcohol or O-acylated derivative having the general structure: 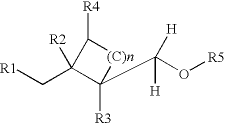
wherein, R1 is H or Me;
R2 is H or Me;
R3 is selected from the group consisting of H, Me, and OMe;
R4 is H or Me;
R5, is selected from the group consisting of H; —C(O)R6, where R6 is selected from the group consisting is selected from Me, Et, Pr, iPr, Bu, and —CH₂cyclopropyl, —CH₂CH₂CH═CH₂, —CH₂OCH₃, —CH₂COCH₂CH₃, —CH₂CH₂OCH₃, —CH₂CH₂OCH₂CH₃, and
n is 0 or 1.

72. The composition of claim 71 wherein said component is selected from the group consisting of

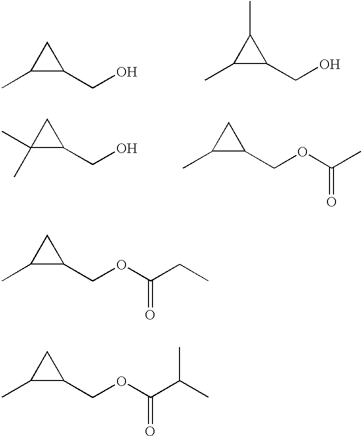

73. The composition of claim 71 which is selected from the group consisting of: a) personal care products including underarm deodorants and antiperspirants, hair shampoos and conditioners, toilet soaps, and skin lotions; b) fabric care products including laundry detergents, fabric conditioners, and fabric fresheners; c) pet care products including deodorant, shampoo, conditioner: and d) home care products including air fresheners, carpet fresheners, surface cleaners and hand wash dish detergents.

74. A composition adopted for blocking or reducing the odor of at least one malodorous carboxylic acid selected from the group consisting of isovaleric acid; 3-methyl-2-hexenoic acid, hexanoic acid, and propionic acid comprising an effective odor blocking or reducing amount of at least one compound selected from the group consisting of (2-methylcyclopropyl)methanol, 2-(2-methylcyclopropyl)ethanol, 2-cyclopropyl ethanol, isoamyl alcohol, 2-(11-methylcyclopropyl)ethanol, isoamylacetate, (2,2-dimethylcyclopropyl)methanol and 3-methylbut-3-enol.

75. The composition of claim 74 wherein said component is (2-methyl-cyclopropyl)methanol.

76. The composition of claim 74 wherein said component is 2-(2-methyl-cyclopropyl)ethanol.

77. The composition of claim 74 wherein said component is 2-cyclopropyl ethanol.

78. The composition of claim 74 which is selected from the group consisting of: a) personal care products including underarm deodorant and antiperspirant, hair shampoos and conditioners, toilet soaps, and skin lotions; b) fabric care products including laundry detergents, fabric conditioners, and fabric fresheners; c) pet care products including deodorants, shampoos, conditioners: and d) home care products including air fresheners, carpet fresheners, surface cleaners and hand wash dish detergents.

79. A method for blocking or reducing the odor of a malodorous carboxylic acid selected from the group consisting of butyric acid, propionic acid, isovaleric acid, hexanoic acid and 3-methyl-2-hexenoic acid comprising contacting an environment or substrate prone to accumulation or contamination of one or more of said malodorous carboxylic acids with an odor reducing effective amount of an ester having the following general structure: 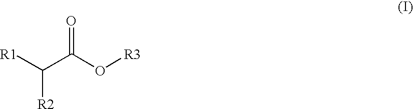
wherein R1 is selected from the group consisting: 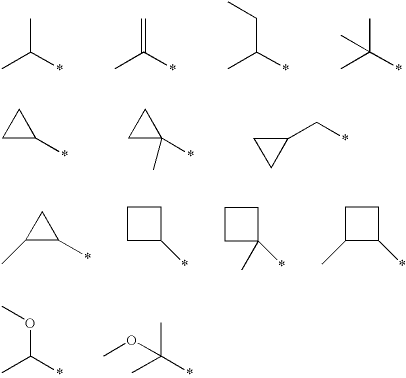
R2 is selected from the group consisting of H, Me, Et, OH, and OMe;
R3 is selected from the group consisting of H, Me, Et, propyl, isopropyl, —CH₂cyclopropyl, —CH₂CH₂CH═CH₂, —CH₂CH₂OCH₃, —CH₂CH₂OCH₂CH₃.

80. The method of claim 79 wherein said compound is selected from the group consisting of

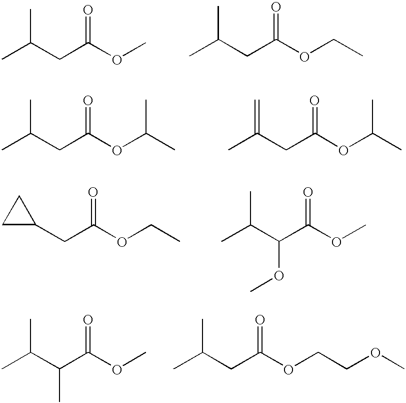

81. The method of claim 79 wherein the substrate is selected from the group consisting of human or animal skin, air, a fabric, and a carpet or rug.

82. The method of claim 79 wherein the malodorous carboxylic acid is isovaleric acid.

83. The method of claim 79 wherein the malodorous carboxylic acid is 3-methyl-2-hexenoic acid.

84. A method for blocking or reducing the odor of a malodorous carboxylic acid selected from the group consisting of butyric acid, isovaleric acid, propionic acid, hexanoic acid and 3-methyl-2-hexenoic acid comprising contacting an environment or substrate prone to accumulation or contamination with one or more of said malodorous carboxylic acids with an ester having the following generic structure: 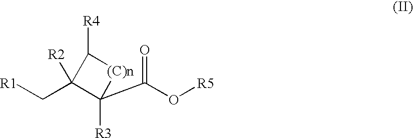
wherein, R1 is H or Me;
R2 is H or Me;
R3 is selected from the group consisting of H, Me, and OMe;
R4 is H or Me;
R5, is selected from the group consisting of H, Me, Et, propyl, isopropyl, —CH₂cyclopropyl, —CH₂CH₂CH═CH₂, —CH₂CH₂OCH₃, —CH₂CH₂OCH₂CH₃, and n is 0 or 1.

85. The composition of claim 84 wherein said component is selected from the group consisting of

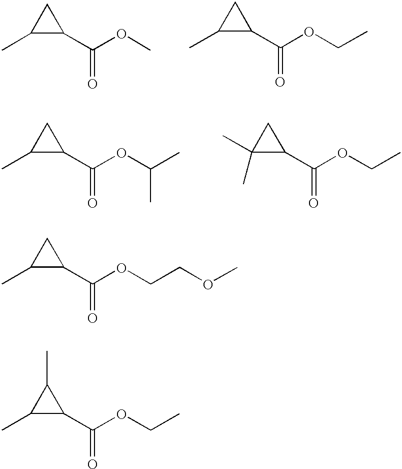

86. The method of claim 84 wherein said substrate is selected from the group consisting of human or animal skin, air, a fabric, and a carpet or rug.

87. The method of claim 84 wherein the malodorous carboxylic acid is isovaleric acid.

88. The method of claim 84 wherein the malodorous carboxylic acid is 3-methyl-2-hexenoic acid.

89. A method for blocking or reducing the odor of a malodorous carboxylic acid selected from the group consisting of isovaleric acid, 3-methyl-2-hexenoic acid, hexenoic acid and propionic acid comprising contacting an environment prone to the accumulation or contamination of one or more of said malodorous carboxylic acids with an effective amount of one ester selected from the group consisting of ethyl isovalerate, methyl isovalerate, 2-methylcyclopropanecarboxylic acid methyl ester, n-propyl isovalerate, 2-methylcyclopropanecarboxylic acid ethyl ester, isopropyl isovalerate, 2-methylcyclopropanecarboxylic acid isopropyl ester, and 2-methylcyclopropanecarboxylic acid n-propyl ester.

90. The method of claim 89 wherein said ester is methyl isovalerate.

91. The method of claim 89 wherein said ester is ethyl isovalerate.

92. The method of claim 89 wherein said ester is 2-methylcyclopropane carboxylic acid methyl ester.

93. A composition adapted for blocking or reducing the odor of at least one malodorous carboxylic acid selected from the group consisting of isovaleric acid, 3-methyl-2-hexenoic acid, hexenoic acid and propionic acid comprising an effective odor blocking or reducing amount of at least one ester having the general structure: 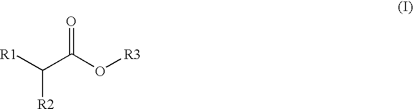
wherein R1 is selected from the group consisting: 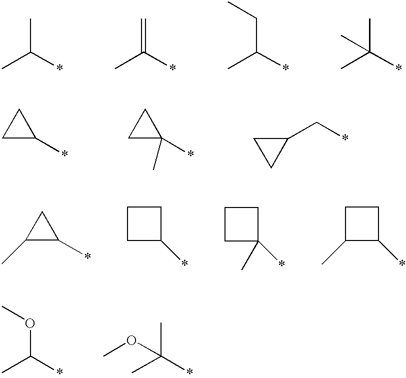
R2 is selected from the group consisting of H, Me, Et, OH, and OMe;
R3 is selected from the group consisting of H, Me, Et, propyl, isopropyl, —CH₂cyclopropyl, —CH₂CH₂CH═CH₂, —CH₂CH₂OCH₃, —CH₂CH₂OCH₂CH₃.

94. The composition of claim 93 wherein said compound is selected from the group consisting of

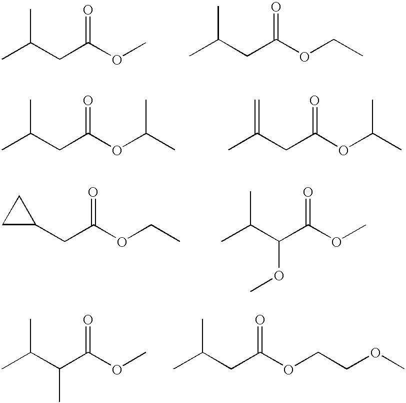

95. The composition of claim 94 which is selected from the group consisting of: a) personal care products including underarm deodorants and antiperspirants, hair shampoos and conditioners, toilet soaps, and skin lotions; b) fabric care products including laundry detergents, fabric conditioners, and fabric fresheners; c) pet care products including deodorants, shampoos, conditioners: and d) home care products including air fresheners, carpet fresheners, surface cleaners and hand wash dish detergents.

96. A composition adapted for blocking or reducing the odor of at least one malodorous carboxylic acid selected from the group consisting of isovaleric acid, 3-methyl-2-hexenoic acid, hexenoic acid and propionic acid comprising an effective odor blocking or reducing amount of at least one ester having the general structure: 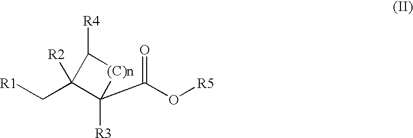
wherein, R1 is H or Me;
R2 is H or Me;
R3 is selected from the group consisting of H, Me, and OMe;
R4 is H or Me;
R5, is selected from the group consisting of H, Me, Et, propyl, isopropyl, —CH₂cyclopropyl, —CH₂CH₂CH═CH₂, —CH₂CH₂OCH₃, —CH₂CH₂OCH₂CH₃, and n is 0 or 1.

97. The composition of claim 98 wherein said component is selected from the group consisting of

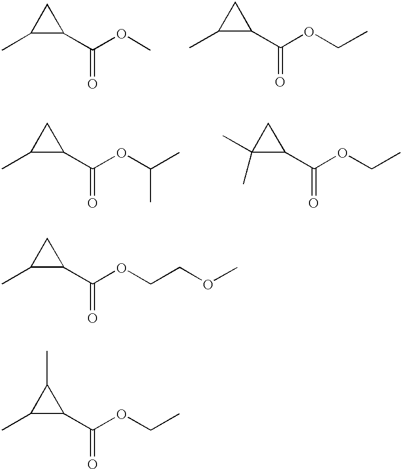

98. The composition of claim 97 which is selected from the group consisting of: a) personal care products including underarm deodorants and antiperspirants, hair shampoos and conditioners, toilet soaps, and skin lotions; b) fabric care products including laundry detergents, fabric conditioners, and fabric fresheners; c) pet care products including deodorants, shampoos, conditioners: and d) home care products including air fresheners, carpet fresheners, surface cleaners and hand wash dish detergents.

99. A composition adopted for blocking or reducing the odor of at least one malodorous carboxylic acid selected from the group consisting of isovaleric acid; 3-methyl-2-hexenoic acid, hexanoic acid, and propionic acid comprising an effective odor blocking or reducing amount of at least one ester selected from the group consisting of ethyl isovalerate, methyl isovalerate, 2-methylcyclopropanecarboxylic acid methyl ester, n-propyl isovalerate, 2-methylcyclopropanecarboxylic acid ethyl ester, isopropyl isovalerate, 2-methylcyclopropanecarboxylic acid isopropyl ester, and 2-methylcyclopropanecarboxylic acid n-propyl ester.

100. The composition of claim 99 wherein said ester is methyl isovalerate.

101. The composition of claim 99 wherein said ester is ethyl isovalerate.

102. The composition of claim 99 wherein said ester is 2-methylcyclopropane carboxylic acid methyl ester.

103. The composition of claim 99 which is selected from the group consisting of: a) personal care products including underarm deodorants and antiperspirants, hair shampoos and conditioners, toilet soaps, and skin lotions; b) fabric care products including laundry detergents, fabric conditioners, and fabric fresheners; c) pet care products including deodorants, shampoos, conditioners: and d) home care products including air fresheners, carpet fresheners, surface cleaners and hand wash dish detergents.
